(19) United States
(12) Patent Application Publication
(10) Pub. No.: US 2006/0210979 A1
(43) Pub. Date: Sep. 21, 2006

(54) Methods of detecting gene expression in normal and cancerous cells

(76) Inventors: Lily Yang, Deeatur, GA (US); Gang Bao, Mableron, GA (US); Charles Staley, Arlanta, GA (US); Cynthia Cohen, Atlanta, GA (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(21) Appl. No.: 10/542,117

(22) PCT Filed: Jan. 13, 2004
(86) PCT No.: PCT/US04/00755

§ 371 Date: Nov. 30, 2005

Related U.S. Application Data
(60) Provisional application No. 60/439,771, filed on Jan. 13, 2003.

(51) Int. Cl. C12Q 1/68

(52) U.S. Cl. 435/6

(57) ABSTRACT

The present invention provides methods for detecting gene expression in normal and cancerous cells. Specifically, provided are methods utilizing molecular beacons (MB) technology combined with fluorescence imaging techniques for detecting, identifying or quantitating the presence of, or alterations in gene expression of, various tumor markers in a sample of cells.

This application is being filed on 13 Jan. 2004, as a PCT International Patent application in the name of Emory University, a U.S. national University, applicant for the designation of all countries except the US, and Lily Yang, Gang Bo, Charles Staley, and Cynthia Cohen

### FIELD OF THE INVENTION

This invention relates generally to methods of detecting human cancer cells through examination of the levels of expression of tumor marker genes and mutant oncogenes in normal and/or cancerous cells using molecular beacon technology.

### BACKGROUND OF THE INVENTION

Breast cancer is the most common type of cancer and a leading cause of death among women. A crucial factor to increase survival is to diagnose it early. Although early screening with mammography decreased the mortality of the disease, nearly 20% of breast cancer patients are still missed by mammography. Furthermore, of all patients with abnormal mammograms, only 10 to 20% were confirmed to be breast cancer by biopsy (Harris et al., In Detita VT, Lippincott-Raven; 1557-1616 (1997)). At present, there is no reliable serum tumor marker for diagnosis of breast cancer. Therefore, development of novel approaches for early diagnosis of breast cancer is of critical importance for the successful treatment and for increasing survival of the patients.

At present, ductal lavage has been used as a minimally invasive procedure to collect breast ductal epithelial cells for cytopathological analysis (O'Shaughnessy et al., Cancer 94(2):292-298 (2002)). This procedure involves inserting a microcatheter into a nipple orifice, lavaging the cannulated duct with normal saline and collecting lavage effluent. About 13,500 cells per duct can be collected for analysis of the presence of normal, atypical, or malignant breast ductal cells. However, the current method for identification of different cell types is by morphological classification which is often inaccurate.

Pancreatic cancer is the fourth leading cause of cancer death in the United States because of its extremely poor prognosis (Parker et al., CA. Cancer J. Clin. 46:5-27 (1996)). About 29,000 new cases are diagnosed and 28,000 of death occur each year in the United States (Gold E. I., Surg. Clin. North Am. 75:819-839 (1995)). Less than 50% of pancreatic patients survive more than three month after diagnosis and 8% of them survive two years (National Institute of Health: NIH Publication 93-2789 (1993)). The main reason for the poor prognosis is that very few of the patients with pancreatic cancers are found early. Early diagnosis of pancreatic cancer using traditional radiographic and ultrasonographic methods is extremely difficult (Barkin et al., Gastroenterology Clinics of North America 28:709-722 (1999)). In spite of the extensive biomedical research efforts during the last few decades, over 90% of the patients with pancreatic cancer have already undergone local and/or distant metastases by the time of diagnosis, often making it too late to cure. Therefore, it is extremely important to have early detection of pancreatic cancer, possibly based on molecular markers rather than the size of the tumor.

Molecular Markers of Pancreatic and Breast Cancer

It has been well established that currently K-ras oncogene is among the most attractive molecular markers for the detection of early pancreatic cancers (Minamoto et al., Cancer Detection & Prevention 24:1-12 (2000); Futakawa et al., Journal of Hepato-Biliary-Pancreatic Surgery 7:63-71 (2000); Puig et al., International Journal of Cancer 85(1):73-77 (2000); Watanabe et al., Pancreas 17:341-7 (1998); Shibata et al., International Journal of Oncology 12:1333-1338 (1998); Urgell et al., European Journal of Cancer 36:2069-2075, 2000)). A member of the G-protein family, K-ras is involved in signal transduction of growth-promoting effectors from the cell surface. Point mutations of K-ras are found in 80 to 100% of pancreatic carcinomas, suggesting that it is a sensitive marker for cancer detection (Minamoto et al., Cancer Detection & Prevention 24:1-12 (2000)). Further, most of these mutations are concentrated at codon 12, making K-ras even more attractive for the ease of beacon design. Since K-ras mutations occur very early in the development of pancreatic cancer, tests targeting K-ras mutations can lead to early detection of pancreatic carcinomas.

Recently, genes of a family of inhibitor of apoptosis proteins (IAPs) have been discovered (LaCasse et al., Oncogene 17:3247-3259 (1998)). IAPs inhibit the cascade of the apoptotic pathway through inhibition of caspase activity (Deveraux et al., EMBO Journal 17(8):2215-2223 (1998)). Increasing evidence indicate that one of the LAP proteins, survivin, is also a promising tumor marker for several types of cancers (Altieri et al., Lab. Invest. 79:1327-1333 (1999); Tanaka et al., Clin. Cancer Res. 6:127-134 (2000); Moore et al., J. of Insurance Med. 33:202-203 (2001); Altieri et al., Trends in Mol. Med. 7:542-547 (2001)). Survivin is normally expressed during fetal development but is not expressed in most normal adult tissues (Altieri et al., Lab. Invest. 79:1327-1333 (1999)). Results from the analysis of 3.5 million transcripts from 19 human normal and diseased tissue types have also revealed that survivin is one of the top four genes that is uniformly expressed at elevated levels in all cancer tissues examined, but not in normal tissues (Velculescu et al., Nature Genetics 23:387-388 (1999)). A recent study has demonstrated the presence of survivin in 77% of pancreatic duct cell adenocarcinoma and 56% intraductal papillary-mucinous tumor (IPMT) by immunohistochemistry, immunoblotting and RT-PCR assays (Saton et al., Cancer 92:271-278 (2001)). Expression of survivin can be detected in all stages (from I to IV) of pancreatic duct cell carcinoma including in early stages of neoplastic transition in pancreatic cancer cells. However, expression of survivin was not detected in normal pancreatic tissues, inflammatory cells around tumor cells and pancreatic tissues from patients with chronic pancreatitis. Absence of survivin expression in normal pancreas and other normal tissues makes it an ideal molecular marker for the detection of pancreatic cancer cells.

The transition from normal mammary epithelial to invasive ductal carcinoma is a multistage process, which involves a series of histological changes in the breast tissues from hyperplasia, atypical hyperplasia to duct carcinoma in situ (DCIS) and to invasive ductal carcinoma. Several tumor markers have been found to be present in DCIS lesions and invasive breast cancers. Cyclin D 1, an important regulator for cell cycle, is overexpressed in 80% of DCIS whereas it is low or absent in normal breast tissues (Weinstat-Saslow et al., Nat Med 1(12):1257-1260 (1995); Vos et al., J. of Path. 187(3):279-84 (1999)). Amplification and overexpression of Her-2/neu gene are also demonstrated in 30% of invasive breast cancers and 60 to 80% of DCIS tissues (Janocko et al., Cytometry 46(3):136-49 (2001); Poller et al., Breast Cancer Res. & Treat. 20(1):3-10(1991); Ramachandra et al., J. of Path. 161(1):7-14 (1990)). However, overexpression of Her-2/neu is not found in normal ductal cells and in simple hyperplasia (Poller et al., Breast Cancer Res. & Treat. 20(1):3-10 (1991); Ramachandra et al., J. of Path. 161(1):7-14 (1990)). This suggests that regulation of these genes may define a transition from a benign state to carcinoma and that unregulated overexpression of cyclin D 1 and Her-2/neu may be a common early event in mammary carcinomas. Additionally, high levels of survivin are also detected in 71% of breast cancer tissues obtained from the patients with invasive and metastatic breast cancers while the surrounding normal breast tissues are negative. An increase in the levels of survivin contributes to a higher apoptotic threshold and survival ability of the breast tumor cells (Tanaka et al., Clin. Cancer Res. 6:127-134 (2000)). Expression of survivin gene appears to be an early tumor marker in developing breast cancer since a high level of survivin is detected in over 80% of DCIS tissues of the breast cancer patients (Yang L. et al. Unpublished observations). Therefore, cyclin D 1, Her-2/neu and survivin are sensitive tumor markers for early detection of breast cancer cells at pre-invasive stage.

Survivin is also detected in many common tumor types such as prostate, lung, colon, gastric, liver, brain, renal, melanoma and lymphoma (Altieri et al., Trends in Mol. Med. 7:542-547 (2001)). For example, 64% of human colorectal cancers express a high level of survivin. Five-year survival rate in the stage II patients with positive survivin are much less than that of the survivin negative patients (Sarela et al., Gut 46(5):645-650 (2000)). The correlation of the survivin expression and prognosis of cancer patients has also been demonstrated in several other tumor types (Kappler et al., International Journal of Cancer. 95:360-363 (2001); Swana et al., New England Journal of Medicine. 341:452-453 (1999)). Several reports indicated that the expression of survivin gene in tumor cells contributed to resistance of the tumor cells to chemo- or radiotherapy (Kato et al., International Journal of Cancer 95:92-5 (2001); Azuhata et al., Journal of Pediatric Surgery 36:1785-1791 (2001); Asanuma et al., Japanese Journal of Cancer Research 91:1204-9 (2000)). Therefore, level of survivin gene expression could be used to determine the sensitivity of the human tumor cells to chemotherapy drugs.

Detecting Human Cancer Cells with the Molecular Beacon Technology

At present, the commonly used methods for detection of the gene mutation in clinical samples are DNA purification of genomic DNA or RNA isolation followed by mutant-enriched PCR or RT-PCR. The presence of mutant PCR products is then determined by single strand conformation polymorphism (SSCP), restriction fragment-length polymorphisms (RELP), or allele-specific oligodeoxynucleotide hybridization (ASOH) (Futakawa et al., Journal of Hepato-Biliary-Pancreatic Surgery 7:63-71 (2000); Puig et al., International Journal of Cancer 85(1):73-77 (2000); Watanabe et al., Pancreas 17:341-7 (1998); Shibata et al., International Journal of Oncology 12:1333-1338 (1998); Fischer et al., Laboratory Investigation 81:827-831 (2001); Clayton et al., Clinical Chemistry 46:1929-1938 (2000)). Although identification of K-ras mutations by PCR is a fairly sensitive molecular approach, the procedures for PCR and subsequent assays are very time-consuming, making them difficult to become clinical procedures. So far, it has been very difficult to directly detect the expression of mutant oncogenes in intact cells since in situ hybridization method with current fluorescence-labeled-linear oligonucleotide probes have a high level of fluorescence background and a low sensitivity in detecting mRNAs with single base pair mutation. Immunostaining with antibodies to mutant proteins usually lacks specificity and generates “false positive” data A high number of “false positives” has been observed due to non-specific labeling and presence of endogenous peroxidase or alkaline phosphatase. Thus, it is important to develop more specific assays for detection of the tumor cells expressing a mutant oncogene.

It is well established that cancer cells develop due to genetic alterations in oncogenes and tumor suppressor genes and abnormalities in gene expression that provide growth advantage and metastatic potential to the cells. A hereunto for utilized method of achieving early detection of cancer would be to identify the cancer cells through detection of mRNA transcripts that are expressed in the cancer cells but is low or not expressed in normal cells. Therefore, a heretofore-unaddressed need exists in the art to address the aforementioned deficiencies and inadequacies.

### SUMMARY OF THE INVENTION

Various aspects of the present invention provide methods for detecting the level of gene expression in fixed or viable normal and cancerous cells. Specifically, provided are methods utilizing molecular beacons (MB) technology combined with fluorescence imaging techniques for detecting, identifying or quantitating the presence of, or alterations in gene expression of, various tumor markers in a sample of cells.

MBs are single-stranded oligonucleotides with a fluorophore at one end and a quencher at the other; they are designed to form a stem-loop structure when their target mRNA is not present such that the fluorescence of the fluorophore is quenched. The loop portion has a probe sequence complementary to a target mRNA molecule. The arm sequences near each end of the loop are complementary to each other; they anneal to form the MB's stem. When the MB encounters a target mRNA molecule, the loop and possibly a part of the stem hybridize to the target mRNA, causing a spontaneous conformational change that forces the stem apart. The quencher moves away from the fluorophore, leading to the restoration of fluorescence. A major advantage of the stem-loop probes is that they can recognize their targets with a higher specificity than the linear probes. Properly designed MBs could discriminate between targets that differ by as little as a single nucleotide. The design of MBs also allows specific binding of the MBs to their target nucleotide sequences and reports the hybridization through generating a fluorescence signal without separation of unbound probes from MB-target complex since free MBs do not fluoresce. Therefore, MBs should provide us with an excellent tool for detecting specific nucleotide sequence, such as mRNA and DNA, with a high noise to signal ratio in intact cells as well as in solution.

Thus, in one aspect, the invention is related to a method of detecting the presence of at least one tumor marker mRNA in a sample. The method includes providing a sample of cells for analysis and then treating the sample with a circle oligonucleotide (MB) that targets the tumor marker mRNA. The hybridization of the target sequence is then detected, identified or quantitated under suitable hybridization conditions, such that the presence, absence or amount of target sequence present in the sample is correlated with a change in detectable fluorescence signal. The presence of a tumor marker can then be detected, identified or quantitated based upon the presence, absence or amount of the hybridization of the oligonucleotide to the target sequence that is determined. The MBs can be delivered into acetone-fixed cells by direct incubation or into viable cells through transfection. The presence and quantification of level of tumor marker mRNAs after delivery of the MBs into fixed or viable tumor cells are accomplished by measuring the fluorescence intensity using a fluorescence microscope, using FACS-scan analysis of individual cell populations or monitoring the changes of the relative fluorescence unit real-time in 96-well plate using a fluorescence microplate-reader.

The tumor marker to be detected and be any tumor marker and in certain aspects of the present invention can include one or more of the following: survivin, cyclin D1, Her2/neu, a mutant K-ras, chymotrypsinogen, XIAP, basic fiborblast growth factor, EGF receptor, carcinoembryonic antigen, prostate, specific antigen, alpha-fetoprotein, beta-2 microglobulin, bladder tumor antigen, chromogranin A, neuron-specific enolase, S-100, TA-90, tissue polypeptide antigen and human chorionic gonadotropin.

In any aspect of the present invention, the sample can be taken from one or more of any number of sources including, but not limited to, blood, urine, pancreatic juice, ascites, breast ductal lavage, nipple aspiration, needle biopsy or tissue. In certain aspects of the invention, the tissue is a biopsy from the pancreas or breast. In other aspects of the invention, the tissue can be in the form of a frozen microscope section.

Another aspect of the present invention related to a method for detecting the presence of a mutant gene in a tumor cell that includes providing a sample of tumor cells for analysis and then treating the sample with an oligonucleotide that targets the mutant gene. In certain aspects of the present invention, the mutant gene is a mutant K-ras gene.

Still another aspect of the present invention relates to a method of monitoring the level of gene expression in viable cells.

Yet another aspect of the invention relates to a method of detecting or monitoring the presence or progression of breast cancer in a subject that includes monitoring or detecting the presence of a breast cancer marker. In various aspects of the invention, the breast cancer marker can be one or more of the following: survivin, cyclin D1, Her2/neu, basic fibroblast growth factor and carcinoembryonic antigen. In certain aspects of the invention, the sample can be taken from, but is not limited to, one or more of the following: blood, urine, breast ductal lavage, nipple aspiration, ascites needle biopsy or tissue.

Still yet another aspect of the present invention relates to a method of detecting or monitoring presence or progression of pancreatic cancer in a subject that includes detecting or monitoring for the presence of a pancreatic cancer marker. In various aspects of the invention, the pancreatic cancer marker can be, but is not limited to, one or more of: survivin, a mutant K-ras gene, and carcinoembryonic antigen. Various aspects of the invention also provide that the sample can be taken from at least one source including, but not limited to, blood, urine, pancreatic juice, needle biopsy or tissue.

Another aspect of the present invention relates to a method of detecting cancerous cells in a sample that includes treating a sample of cells with an oligonucleotide that targets a cancer-specific marker gene sequence. In various aspects of the invention, the cancer cell can originate from one or more of the following cancers, including but not limited to, breast, pancreas, ovarian, prostate, colorectal, hepatocellular, multiple myeloma, lymphoma, bladder, medullary carcinoma of the thyroid, neuroendocrine tumors, carcinoid tumors, testicular, gestational trophoblast neoplasms, lung, melanoma and stomach.

Other aspects of the present invention provide diagnostic kits for 1) detecting or monitoring the progression of cancerous cells; and, 2) detecting the level of gene expression in viable cells in real-time.

### BRIEF DESCRIPTION OF THE DRAWINGS

FIG. 1 depicts a schematic illustration of molecular beacons (MBs). (A) Molecular beacons are dual labeled oligonucleotides with a hairpin structure; (B) delivering molecular beacons into cells can result in a fluorescent signal due to hybridization of the probe with a target mRNA.

FIG. 2 shows specific binding of molecular beacon probes to their oligonucleotide targets in vitro. K-ras MB1, K-ras MB2, survivin and cyclin D1 MBs were mixed with synthesized specific or non-specific DNA targets and incubated for 1 hour at 37° C. (survivin and cyclin D1) or 50° C. (K ras). The relative fluorescence unit (RFU) was measured in a fluorescence microplate reader. The bar in the figure is the mean RFU of four repeat samples. FIGS. 2A and B: K-ras MB 1 or MB 2 specifically bound to K-ras Mut 1 or Mut 2 target resulting in a higher fluorescence intensity as compared with K-ras WT target or the target with a different k-ras mutation. FIGS. 2C and D. Survivin MB or cyclin D1 MB hybridized only to survivin or cyclin D1 target but do not bind to K-ras or Her-2 target resulting in a high level of fluorescence signal.

FIG. 3 depicts the molecular beacon imaging of pancreatic cancer cells by simultaneous detection of expression of mutant K-ras and survivin genes in human Pancreatic cancer cell lines. Pancreatic cancer and normal cell lines were fixed with acetone. The cells were incubated with mixtures of K-ras MB 1-cy 3 (GGT to GAT) with survivin MB-FITC or K-ras MB2-Texas red (GGT to GTT) with survivin MB-FITC for 1 hour at 50° C. The slides were then observed under a confocal fluorescence microscope. The fluorescent images were taken and same exposure condition was used to take all images for each color. The cells with red fluorescence were pancreatic cancer cells expressing specific mutant K-ras as detected either by K-ras MB 1 or K-ras MB 2. The cells were also double labeled with green fluorescence showing that these tumor cells also expressed a tumor marker survivin. FIG. 3A shows the specificity of detection of pancreatic cancer cells expressing mutant K-ras and survivin genes in the tumor cell lines with specific K-ras mutations and a high level of survivin. Panc-1 cell line has a K-ras GGT to GAT mutation and showed strong fluorescence intensity after incubating with K-ras MB 1, but displayed a weak fluorescence in K-ras MB2-stained cells. Capan-2 cell line contains a K-ras GGT to GTT mutation and a brighter fluorescence was detected in K-ras MB 2 stained cells. Both cell lines expressed a high level of survivin as detected by survivin MB. FIG. 3B shows pancreatic cancer cells expressing a different mutant or wild type K-ras gene showed a weak or negative for K-ras MBs but can still be detected by survivin MB. K-ras MBs did not produce strong fluorescence signaling in MIA PaCa-2 cell line, which has a K-ras GGT to TGT mutation, or K-ras wild type BXPC-3 cells. However, those cells were positive for survivin MB staining. Incubation of K-ras and survivin MBs did not produce detectable fluorescence signaling in a normal cell line (HDF), which is generated from normal dermal fibroblasts. FIG. 3C shows the comparison of fluorescence intensity of pancreatic cancer and normal cells after delivery of K-ras MBs. The fluorescent intensity was measured in each image at three randomly selected areas using Adobe Photo Shop software. The numbers in the bar figure represent the mean fluorescence intensity of 5 to 7 images.

FIG. 4 depicts molecular beacon imaging of human breast cancer cells expressing tumor markers cyclin d1 and survivin. FIG. 4A shows breast cancer and normal mammary epithelial lines that were incubated with a mixture of cyclin D 1-texas red and survivin MB-Alexa 488 for 1 hour at 37° C. The slides were then observed under a confocal fluorescence microscope. The fluorescent images were taken and same exposure condition was used to take all images for each color. FIG. 4B shows levels of fluorescence intensity in breast cancer cell lines detected by survivin and cyclin D1 MBs are correlated the levels of survivin and cyclin D1 proteins detected by Western blot analysis.

FIG. 5 shows the detection of survivin expression on frozen sections of human breast cancer tissues at DCIS and invasive stages by immunofluorescence staining for survivin protein or survivin mb detecting survivin mRNA. Frozen tissue sections of breast cancer or normal tissues were incubated with either a polyclonal survivin antibody or survivin MB-cy3. FIG. 5A. Survivin is early tumor marker and both survivin mRNA and protein could be detected in the early stage of breast cancer, duct carcinoma in situ (DCIS). Survivin is also detected in invasive breast cancer but not in normal breast tissues. FIG. 5B. Survivin MB detected metastatic breast cancer cells in the lymph node, indicating the feasibility of development of a quick and sensitivity method for detecting the presence of breast cancer cells in the lymph node of the patients.

FIG. 6 depicts specific imaging of pancreatic cancer cells expressing mutant K-ras and survivin mRNAs on frozen tissue sections of pancreatic cancer tissues. Frozen tissue sections were incubated with K-ras MB 1 or K-ras MB 2, or survivin MB for 1 hour and counterstained with Hoechst 33342 (blue) FIG. 6A: K-ras MB 1 detected the cancer cells expressing a GGT to GAT mutant K-ras gene on the frozen sections of pancreatic cancer tissues from patient #1 and #2, which contained K-ras codon 12 GGT to GAT mutation. However, bright red fluorescent cells were found on frozen sections of pancreatic cancer tissues from patient #5, which had a K-ras GGT to GTT mutation after incubation with K-ras MB 2 but not with K-ras MB1. FIG. 6B: Detection of the levels of survivin protein or mRNA in pancreatic cancer cells on frozen tissue sections by immunofluorescence or survivin MB staining. For detection of the levels of survivin protein by immunofluorescence staining, frozen tissue sections of pancreatic cancer and normal tissues were incubated with a mouse anti-survivin antibody (survivin Ab) followed by a FITC-labeled goat anti-mouse secondary antibody. High levels of survivin protein and mRNA were found in pancreatic cancer tissues but not in the normal pancreatic tissues.

FIG. 7 shows the real-time detection of survivin gene expression in viable breast cancer cells after EGF treatment. FIG. 7A: breast cancer cell line MCF-7 was cultured in 96-well plate and placed in the medium containing 2% FBS for overnight. The cells were then transfected with a mixture of 200 nM of survivin-6 FAM and GAPDH-cy3 MBs using lipofectamine 2000 for three hours. 200 ng of EGF was then added to the medium. The tissue culture plate was placed immediately in a fluorescence microplate reader and the relative fluorescence unit (RFU) was measured every 30 minutes for three hours. The curve in the figure represents the mean number of three repeat samples and is a ratio of RFU of 6-FAM (survivin MB) and cy3 (internal control GAPDH MB). FIG. 7B: The transfected cells treated with or without EGF were observed at 24 hrs under a confocal microscope and the fluorescence images were taken.

FIG. 8 shows the real-time detection of changes of survivin gene expression using survivin MB at different time points following docetaxel treatment. FIG. 8A: Human breast cancer cell lines MCF-7 and MDA-MB-231 were cultured in 96 well plate. After transfecting with 100 nM of survivin and GAPDH MB mixture, the cells were treated with or without chemotherapy drug docetaxel and the changes of fluorescence intensity in each group were measured real time in a fluorescence microplate reader for 48 hours. FIG. 8B: The transfected cells treated with or without 20 (MDA-MB-231) or 50 nm (MCF-7) of docetaxel for 24 to 48 hrs and then observed under a confocal microscope and the fluorescence images were taken.

FIG. 9 depicts the level of survivin gene expression in viable cells could be detected by FACscan analysis. Breast cancer cell line MDA-MB-231 and normal cell line HDF were transfected with survivin or β-actin MB for 3 hours. The cells were collected from culture dishes and analyzed with FACScan for the level of survivin gene expression in normal and tumor cells. The result demonstrated that the level of gene expression in viable cells could also be measured quantitatively using FACScan.

FIG. 10 shows the detection of the levels of survivin in breast normal and cancer tissues by Western blot analysis. Levels of survivin in paired or non-paired normal and cancer tissue samples from the breast cancer patients.

### DETAILED DESCRIPTION OF THE PREFERRED EMBODIMENTS

Various embodiments of the invention are now described in detail. As used in the description herein and throughout the claims that follow, the meaning of “a,”“an,” and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

Definitions

“Hybridization” and “complementary” as used herein, refer to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary or hybridizable to each other at that position. The oligonucleotide and the DNA or RNA hybridize when a sufficient number of corresponding positions in each molecule are occupied by nucleotides which can hydrogen bond with each other. It is understood in the art that the sequence of an antisense oligonucleotide need not be 100% complementary to that of its target nucleic acid to hybridize thereto. An oligonucleotide is specifically hybridizable when binding of the compound to the target DNA or RNA molecule, and there is a sufficient degree of complementarity to avoid non-specific binding of the antisense oligonucleotide to non-target sequences under conditions in which specific binding is desired, e.g., under physiological conditions in the case of in vivo assays or therapeutic treatment, or, in the case of in vitro assays, under conditions in which the assays are performed.

In the context of this invention, the term “oligonucleotide” refers to an oligomer or polymer of ribonucleic acid (RNA) or deoxyribonucleic acid (DNA) or mimetics thereof. This term includes, but is not limited to, oligonucleotides composed of naturally occurring and/or synthetic nucleobases, sugars, and covalent internucleoside (backbone) linkages. Such modified or substituted oligonucleotides are often preferred over native forms because of desirable properties such as, for example, enhanced cellular uptake, enhanced affinity for nucleic acid targets, and/or increased stability in the presence of nucleases.

The present invention provides methods for detecting gene expression in normal and cancerous cells. Specifically, provided are methods for detecting, identifying or quantitating the presence of, or alterations in gene expression of, various tumor markers in a sample of cells.

Inventors have developed a molecular beacon (MB) technology to detect gene expression in viable as well as fixed tumor cells. MBs are oligonucleotides with a stem-loop hairpin structure, dual-labeled with a fluorophore at one end and a quencher at the other. Delivering MBs into cells will result in a fluorescence signal if the MBs hybridize to target mRNAs. Thus, when the target mRNAs correspond to the molecular markers of a cancer, cancer cells (bright) can be distinguished from normal cells (dark). Methods are provided for: detecting the presence of at least one tumor marker mRNA in a sample of cells; detecting the presence of a mutant gene in a tumor cell; monitoring alterations in gene expression in viable cells; detecting or monitoring the presence or progression of breast cancer in a subject that includes monitoring or detecting the presence of a breast cancer marker; detecting or monitoring presence or progression of pancreatic cancer in a subject that includes detecting or monitoring for the presence of a pancreatic cancer marker; and, detecting cancerous cells in a sample that includes treating a sample of cells with an oligonucleotide that targets a cancer-specific marker gene sequence. Diagnostic kits are also provided that 1) detect or monitor the progression of cancerous cells; and, 2) detect alterations in gene expression in viable cells in real-time.

Molecular Beacons

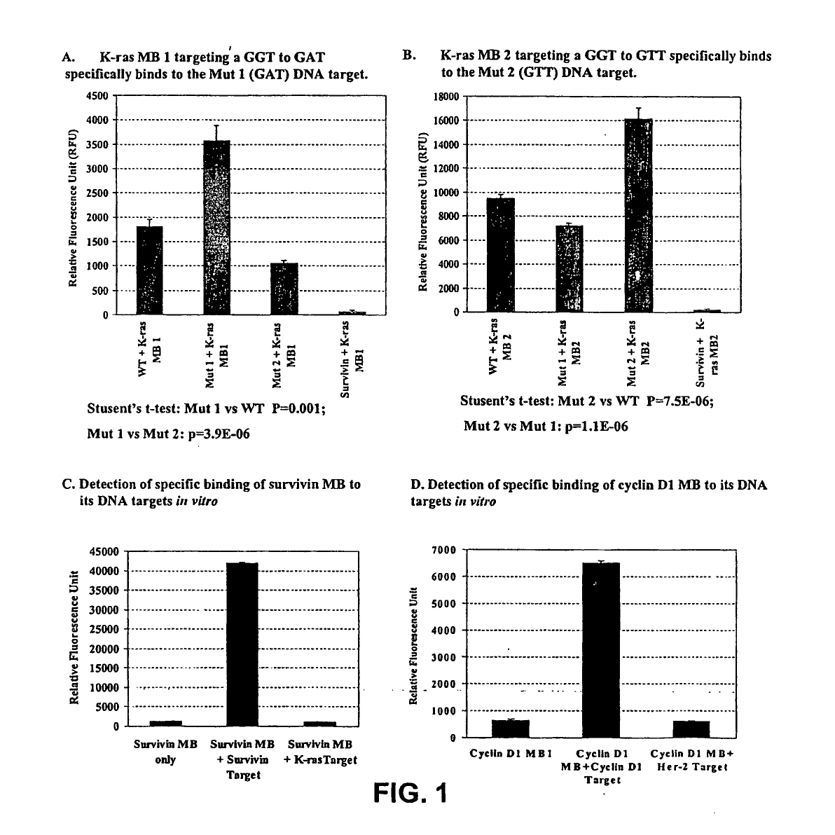

As outlined in FIG. 1, MBs are single-stranded oligonucleotides with a fluorophore at one end and a quencher at the other. Although by no means limiting, typically, the fluorophore is attached to the 5′ end while the quencher is attached to the 3′ end. They are designed to form a stem-loop structure when their target mRNAs are not present such that the fluorescence of the fluorophore is quenched. The loop portion has a probe sequence complementary to a target mRNA molecule. Typical fluorophores that are contemplated to be used include, but are not limited to, Cy3 fluorophore (Cy3 Amidite, Amersham Pharmacia Biotech, Piscataway, N.J.) Alexa Fluor 488 (Molecular Probes); Alexa Fluor 350 (blue), CMAC (7-amino-4-chloromethylcoumarin), 6-FAM and FITC. Typical quenchers include, but are not limited to, dabcyl(4-(4′-dimethylaminophenylazo)benzoic acid) (Dabcyl-CPG, Glen Research, Sterling, Va.).

When the MB encounters a target mRNA molecule, the loop and a part of the stem hybridize to the target mRNA, causing a spontaneous conformational change that forces the stem apart. The quencher moves away from the fluorophore, leading to the restoration of fluorescence (Tyagi et al., Nature Biotechnol 14:303-308 (1996); Dubertret et al., Nat Biotechnol 19:365-370 (2001)). One major advantage of the stem-loop probes is that they can recognize their targets with a higher specificity than the linear oligonucleotide probes. Properly designed MBs can discriminate between targets that differ by as little as a single nucleotide (Tyagi et al., Nat Biotechnol 16:49-53 (1998)). The MBs have been utilized in a variety of applications including DNA mutation detection, protein-DNA interactions, real-time monitoring of PCR, gene typing and mRNA detection in living cells (Dubertret et al., Nat Biotechnol 19:365-370 (2001); Dirks et al., Histochem. & Cell Biol. 115(1):3-11 (2001); Tyagi et al., Nat Biotechnol 16:49-53 (1998); Sokol et al., Proc Natl Acad Sci USA 95:11538-11543 (1998)).

The various embodiments of the present invention provide methods for: detecting the presence of at least one tumor marker mRNA in a sample of cells; detecting the presence of a mutant gene in a tumor cell; monitoring alterations in gene expression in viable cells; detecting or monitoring the presence or progression of breast cancer in a subject that includes monitoring or detecting the presence of a breast cancer marker; detecting or monitoring presence or progression of pancreatic cancer in a subject that includes detecting or monitoring for the presence of a pancreatic cancer marker; and, detecting cancerous cells in a sample that includes treating a sample of cells with an oligonucleotide that targets a cancer-specific marker gene sequence, such methods comprising:
i) providing a sample of cells for analysis;
ii) treating the sample with an oligonucleotide that targets the desired marker or gene;
iii) detecting, identifying or quantitating the hybridization of the target sequence under suitable hybridization conditions, wherein the presence, absence or amount of target sequence present in the sample is correlated with a change in detectable signal associated with at least one donor or acceptor moiety of the oligonucleotide; and
iv) detecting, identifying or quantitating the presence of a marker or gene based upon the presence, absence or amount of the hybridization of the oligonucleotide to the target sequence that is determined.

It is contemplated by the inventors that any oligonucletode constructed for the use as a molecular beacon as described above can be used in methods of the present invention. In particular embodiments, the olignucleotide includes, but is not limited to, one or more of SEQ ID NOS:1, 2, 3, 4, 5, 6, 7, 8, 9, 10, 11, 12, and 13. In one embodiment, the oligonucleotide targets the tumor marker survivin. In a preferred embodiment, the oligonucleotide that targets survivin includes, but is not limited to, one or more of SEQ ID NOS: 1, 2, and 9. In another embodiment, the oligonucleotide targets the tumor marker cyclin D1. In a preferred embodiment, the oligonucleotide that targets cyclin D1, includes, but is not limited to, one or of SEQ ID NOS: 3 and 4. In another embodiment, the oligonucleotide targets the tumor marker Her2/neu. In a preferred embodiment, the oligonucleotide that targets Her2/neu includes, but is not limited to, one or more of SEQ ID NOS: 5 and 6. In yet another embodiment, the oligonucleotide targets the K-ras mutant gene tumor marker. In a preferred embodiment, the oligonucleotide that targets the K-ras mutant gene includes, but is not limited to, one or more of SEQ ID NOS: 7, 8, 11, 12 and 13.

Detection of Tumor Markers

In various embodiments, the present invention provides a method of detecting the presence of at least one tumor marker mRNA in a sample using the molecular beacon technology described herein. As one so skilled in the art will readily appreciate, the methods of the present invention can be utilized to detect the presence of any tumor marker mRNA present in a sample. Such markers include but are not limited to, survivin, cyclin D1, Her2/neu, a mutant K-ras, basic fibroblast growth factor, EGF receptor, XIAP, carcinoembryonic antigen, prostate, specific antigen, alpha-fetoprotein, beta-2-microglobulin, bladder tumor antigen, chromogranin A, neuron-specific enolase, S-100, TA-90, tissue polypeptide antigen and human chorionic gonadotropin.

Sample Collection: Samples of cells to be tested can be obtained through routine diagnostic procedures. Such samples can include, but are not limited to, blood, urine, fine needle aspirates, breast ductal lavage, pancreatic juice, ascites, nipple aspiration samples, or any other tissue, including, but not limited to, a biopsy from anywhere from the patient, including, but not limited to, the breast, the pancreas or a lymph node. The tissue sample can be any type of routine pathologically prepared sample including any type of tissue affixed to a microscope slide, plate or well. In a preferred embodiment, the tissue sample is a frozen section of tissue. In another preferred embodiment, the sample is taken from a breast ductal lavage. In still another preferred embodiment, the sample is taken from pancreatic juice.

Detection of Breast Cancer Tumor Markers

Certain embodiments of the present invention provide a method of detecting or monitoring the presence or progression of breast cancer in a subject using the molecular beacon technology described herein. The present invention provides methods for detecting breast cancer cells from ductal lavage and fine needle aspiration (FNA) using a combination of MBs targeting the mRNAs of genes that have been shown to be expressed in the early stage of tumorigenesis in breast cancer. The inventors have shown the predictive values of the detection of each gene or monitoring the co-expression of two or three genes in the diagnosis of ductal carcinoma in situ. In certain embodiments of the present invention, methods are provided that simultaneously detect of the overexpression of survivin, cyclin D 1 and Her-2/neu genes in breast ductal epithelial cells. The methods of the invention are useful to detect the presence of these tumor markers especially when a tumor single cell expresses more than one marker gene. In a preferred embodiment, a method is provided for the detection of survivin, cyclin D1 and/or Her-2/neu expressing cells in the ductal lavage.

In various embodiments, the methods utilize MBs designed to specifically hybridize to mRNAs of survivin, cyclin D 1 or Her-2/neu. Applicants have demonstrated the specificity and sensitivity of the MBs in human breast cancer cell lines, normal mammary epithelial and normal fibroblast cell lines as well as in identifying the isolated tumor cells in a background of normal cells with different cancer- to normal-cell ratios. The provided methods can be utilized to detect the expression of these tumor markers in the cellular fractions of ductal lavage and aspirates of fine needle aspirates obtained from early stage breast cancer or ductal carcinoma in situ (DCIS) patients at different stages of the disease and normal control subjects.

Sample Collection: Samples of cells to be tested can be obtained through routine diagnostic procedures. Such samples can include, but are not limited to, blood, urine, fine needle aspirates, breast ductal lavage, ascites, nipple aspiration samples, or any other tissue, including, but not limited to, a biopsy from anywhere on the breast or a lymph node. The tissue sample can be any type of routine pathologically prepared sample including any type of tissue affixed to a microscope slide, plate or well. In a preferred embodiment, the tissue sample is a frozen section of tissue.

It is contemplated that samples can be obtained through any routine diagnostic procedure for breast cancer patients or women at a high risk for developing breast cancer. As a non-limiting example, a ductal lavage from a cancer patient is collected when the patient is undergoing surgery to remove the breast cancer or in a routine visit to doctor's office. Under anesthesia, a microcathether is inserted into the duct and saline infused. The effluent fluid is collected and cellular fraction enriched by centrifugation. The enriched cell fraction from ductal lavage or aspirates are then placed on glass slides. Typically, about 10 to 15 cytospin slides are obtained from one ductal lavage with a median of 13,500 epithelial cells per duct. After fixing the cells in ice-cold acetone, the slides are incubated with one or more MBs, either sequentially or simultaneously, at optimized incubation conditions and then examined under a fluorescence microscope. Since MBs for each gene are labeled with different fluorescent dyes, the number of the cells over-expressing any or all of the genes for the target tumor markers in a sample are determined.

Analysis: The inventors contemplate that results obtained from one type of sample collection can be compared with those obtained from another in order to aid in the identification, monitoring or detection of a tumor marker or in the diagnosis of, or monitoring the progression of the cancer. Subsequently, the same slides can be stained and analyzed by a cytopathologist for the presence of benign, atypical or malignant cells. Such staining can include routing cytological stains such as H&E or immuostaining with specific antibodies.

Quantification: Detection of the hybridization of an MB with its target sequence in intact cells, either fixed or viable, can be accomplished by fluorescence microscopy, FACS analysis and fluorescence microplate reader. As a non-limiting example, the breast cancer cells were co-transfected with survivin and GAPDH MBs and then treated with EGF. The alternation of survivin gene expression after EGF treatment was monitored real time in microplate reader for 3 hours. The results showed that EGF induced an increase in survivin gene expression within 30 minutes of the treatment.

Detection of Pancreatic Cancer Tumor Markers

Certain embodiments of the present invention provide a method of detecting or monitoring the presence or progression of pancreatic cancer in a subject using the molecular beacon technology described herein.

One of the crucial issues for early detection of pancreatic cancer is to develop assays that are capable of identifying a few tumor cells in a pool of a large number of normal cells. At present, RT-PCR is the most sensitive assay for detection of the genes that are highly expressed in tumor cells or for mutated gene products such as mutant K-ras gene, or carcinoembryonic antigen. Though RT-PCR can detect one tumor cell in 10⁴ to 10⁵ cells, such assays may generate ‘false positives’. In addition, using current molecular markers, RT-PCR detection of gene expression or mutant gene in peripheral blood and pancreatic juice cannot localize the cancer to pancreas since many types of cancers as well as other non-malignant diseases may also express those molecular markers. Further, RT-PCR assays are very time consuming, typically detecting one gene at a time, making it difficult to become an efficient clinical procedure for cancer diagnosis.

The inventors have developed a sensitive and more efficient method as disclosed herein that can identify a small number of pancreatic cancer cells in peripheral blood and pancreatic juice samples. The MB-based methods disclosed herein can be used to detect pancreatic cancer or tumor cells from a mixed cell population using a single MB type or a combination of several MBs. The methods provided can be used to detect K-ras mutations after RT-PCR amplification of K-ras exon 1. In addition, various cytological or immunostaining procedures can be used in conjunction with the disclosed methods.

For early detection of pancreatic cancer in the high-risk patient population or patients suspected to have pancreatic cancer, it is important to develop clinical assays from patient samples that can be obtained non-invasively or by a minimally invasive procedure. Increasing evidence has revealed the presence of disseminated tumor cells in blood, bone marrow and peritoneal cavity of pancreatic cancer patients (LaCasse et al., Oncogene 17:3247-3259 (1998); Li et al., Nature 396:580-584 (1998); Tamm et al., Cancer Research 58:5315-5320 (1998); Ambrosini et al., Nature Medicine 3:917-921 (1997)). For example, by immunohistochemical staining using antibodies detecting several cytokeratin and tumor markers, the cancer cells were found in 28% of the blood samples obtained from patients with pancreatic cancers. The prevalence of finding cancer cells in blood samples increased with tumor stage. However, it failed to find cancer cells in the blood samples of stage 1 pancreatic cancer patients (Z'graggen et al., Surgery 129:537-545 (2001)). The present invention provides sensitive methods in which various samples from pancreatic cancer patients of all stages can be examined using the MB technology disclosed herein to identify cells expressing pancreatic tumor markers including, but not limited to, mutant K-ras and survivin genes.

Sample Collection: Samples of cells to be tested can be obtained through routine diagnostic procedures. Such samples can include, but are not limited to, blood, urine, fine needle aspirates, pancreatic juice, or any other tissue, including, but not limited to, a biopsy from the pancreas or surrounding tissue. The tissue sample can be any type of routine pathologically prepared sample including any type of tissue affixed to a microscope slide, plate or well. In a preferred embodiment, the tissue sample is a frozen section of tissue. Pancreatic juice can be obtained from patients undergoing diagnostic ERCP procedure. It can also be collected non-invasively from asymptomatic individuals or in high-risk populations by secretin stimulation and sampling of pancreatic juice using duodenoscope. Fine needle biopsy samples from pancreatic cancer patients or pancreatic tumor tissues can be collected after surgery if it is a resectable tumor.

Analysis: The inventors contemplate that results obtained from one type of sample collection can be compared with those obtained from another in order to aid in the identification, monitoring or detection of a tumor marker or in the diagnosis of, or monitoring the progression of the cancer. Subsequently, the same slides can be stained and analyzed by a cytopathologist for the presence of benign, atypical or malignant cells. Such staining can include routing cytological stains such as H&E or immuostaining with specific antibodies.

The sensitivity of the MB-based detection using blood and pancreas juice is evaluated by comparing the size and stages of the pancreatic cancer lesions diagnosed by imaging technologies such as helical CT, MRI or endoscopic ultrasound or pathological diagnosis after surgical resection of the cancer.

Monitoring the Presence of a Mutant Gene

The present invention also provides methods for detecting the presence of a mutant gene in a tumor cell using the molecular beacon technology disclosed herein. It is contemplated that the monitoring for the presence of such mutated genes such as the K-ras gene, will aid in the diagnosis and treatment of various types of cancers, including but not limited to pancreatic cancer.

Monitoring Alterations in Gene Expression

A method of monitoring alterations in gene expression in viable cells in real time using the molecular beacon technology disclosed herein. Such methods will allow for the monitoring expression of target genes including, but not limited to, tumor markers, mutant genes or the like. Thus, clinicians will be able to detect and monitor the development of cancers in for example, individuals who have been determined to be genetically predisposed to certain cancers. In this way, proper treatments can be implemented early in the course of the development of the disease, which may indeed prevent or diminish the onset of tumor or cancer growth. Importantly, detection of the level of gene expression in viable cells will allow one to measure the changes of gene expression real-time in the same cell population after various treatments. This approach can be used for the examination of alternation of gene expression in tumor cells by biological reagents as well as for the evaluation of the expression of molecular target genes after treatment of the cancer cells with therapeutic reagents.

A diagnostic kit for detecting alterations in gene expression in viable cells in real-time is also contemplated that would include any materials or reagents suitable for carrying out the disclosed methods.

Detection of Cancerous Cells

The present invention provides a method of detecting cancerous cells in a sample using the molecular beacon technology disclosed herein. It is contemplated that cancer cells can be detected that originate from one or more of the cancers including but not limited to, breast, pancreas, ovarian, prostate, colorectal, hepatocellular, multiple myeloma, lymphoma, bladder, medullary carcinoma of the thyroid, neuroendocrine tumors, carcinoid tumors, testicular, gestational trophoblast neoplasms, lung, melanoma and stomach. A diagnostic kit for detecting cancerous cells is also contemplated that would include any materials or reagents suitable for carrying out the disclosed methods.

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as data are processed, sampled, converted, or the like according to the invention without regard for any particular theory or scheme of action.

### EXAMPLES

### Example 1

Preliminary Detection of Survivin Gene Expression in Breast Cancer Cell Lines Using Survivin MB

MBs specific for human survivin gene were designed and synthesized, with the probe sequence complementary to the cDNA sequence between 27 nt to 43 nt of the gene (5′-Alexa-fluo 488-CTGAGAAAGGGCTGCCAGTCTCAG-Dabcyl-3′; SEQ ID NO:1). The underlined stem sequences of survivin MB is specially designed to achieve the best thermodynamic effect. At first, specific hybridization of survivin MB was studied with a synthesized survivin oligonucleotide target in vitro.

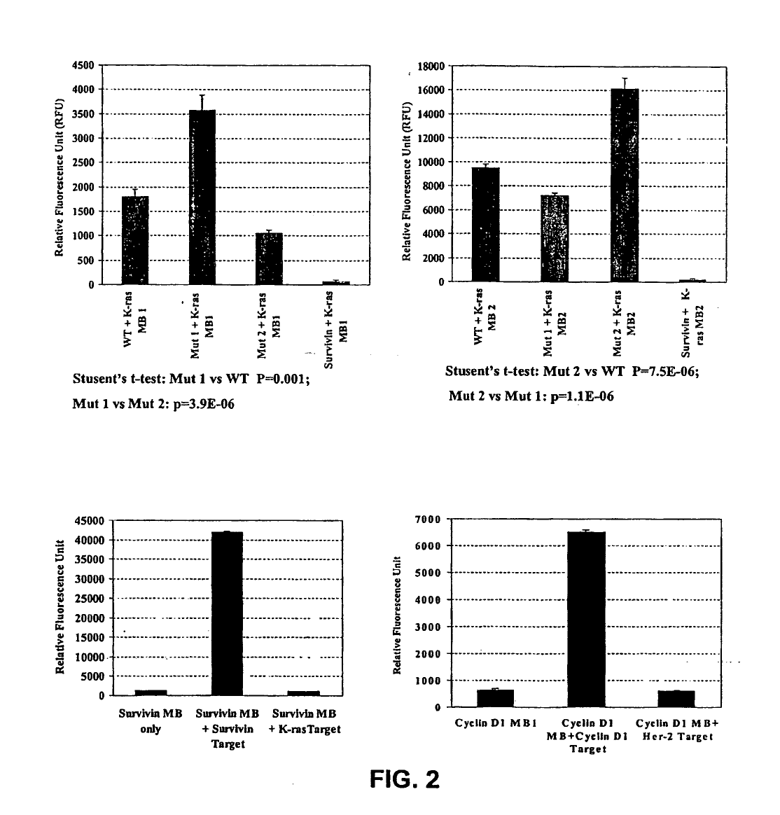

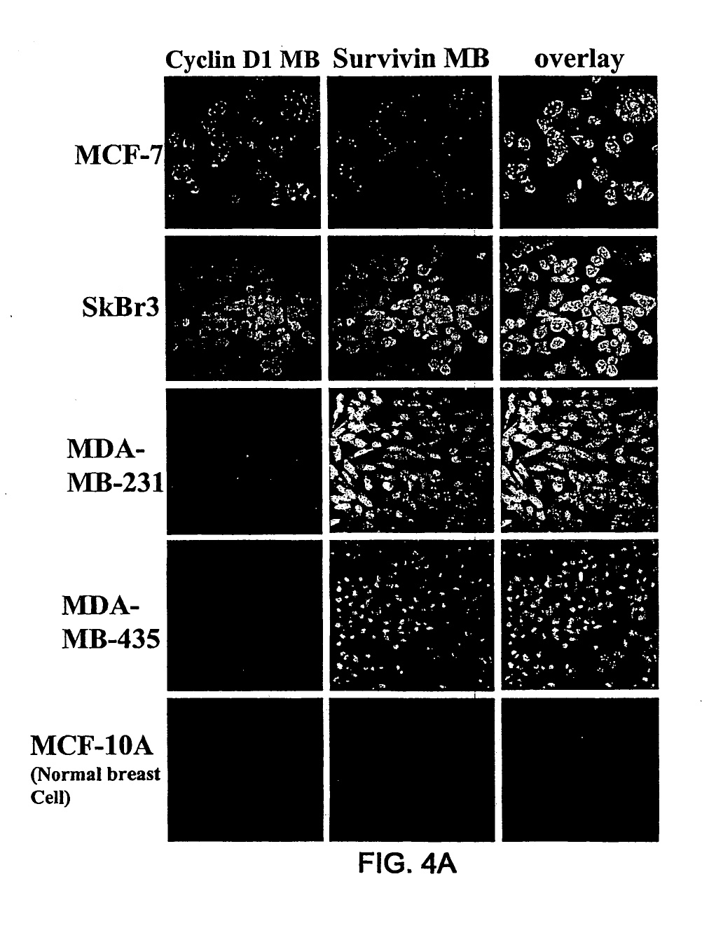

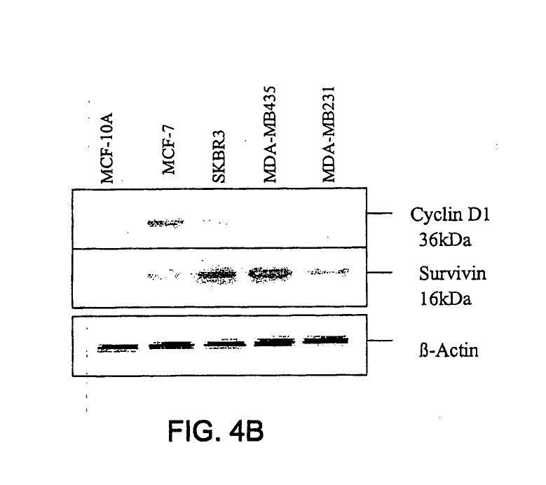

Results indicated that the survivin MB binds specifically to survivin targets (FIG. 2 C). The specificity of survivin MBs for detecting survivin mRNA was further examined in the breast cancer cell lines MDA-MB-231, MDA-MB-435 and MCF-7 expressing different levels of survivin gene and in normal human mammary epithelial cell line (MCF-10A). After incubation of survivin MBs with fixed cells at 37° C. for 1 hour, the cells were washed with PBS and observed under Nikon fluorescence microscope. Cells were grown on chamber slides and fixed with acetone; survivin MB was labeled with Alexa-Fluo 488 (Green, FIG. 4 A). The detection of survivin expression in breast cancer and normal cell lines by Western blot is shown in FIG. 4 B. As shown in FIG. 4 A, tumor cells display from intermediate to strong red fluorescence while in MCF-10A cells, only a weak fluorescence was observed. Furthermore, the intensity of the fluorescence is correlated well with the level of survivin detected by Western blots (FIG. 4 B).

### Example 2

Simultaneous Detection of Expression of Survivin and Cyclin D 1 in Breast Cancer Cells

Further studies were carried out to examine the specificity of detection of cancer cells with several tumor marker MBs. MBs for cyclin D 1 and Her-2/neu genes were designed and synthesized (Table 1). Inventors then examined the specificity of the MBs in vitro with synthesized olignucleotide targets. The sequences for each MB are shown in the Table 1. The underlined sequences are not part of the gene and are designed to form the stem. Inventors have also synthesized a several survivin MB with the same sequences as with different fluorescence dyes (6-FAM, Cy3, Alexa-Fluo-488). Survivin MB-FITC has a different target sequence as shown in the Table 2. The MBs were synthesized by MWG Biotech (High point, N.C.) and Integrated DNA Technologies, Inc. (IDT, Coralville, Iowa).

To determine specific hybridization of the MBs with their targets, hybridization studies were carried out by mixing the MBs with their oligonucleotide targets and then the fluorescence intensities were measured by a fluorescence microplate reader. Control groups for this study were the MBs mixed with oligonucleotide targets from a different gene (FIGS. 2C and D). After demonstration of specific binding of the MBs with their targets, the specificity of the MBs in detection of cyclin D 1 in human breast tumor and normal cell lines was further examined (FIG. 4).

TABLE 1
Molecular beacons for detection of the survivin,
cyclin D 1 and Her-2/neu mRNA

| | | Excitation and |
|---|---|---|
| | | Emission of |
| | | fluorescence dye |
| Genes | MB design | (Color) |
| Survivin | Survivin mRNA (27 to 43 nt) | Ex 491 nm |
| | 5′-Alexa Fluo 488- | Em 515 nm (Green) |
| | CTGAGAAAGGGCTGCCAGTCTCAG-Dabcyl-3′ | |
| | (SEQ ID NO:2) | |
| Cyclin D 1 | Cyclin D 1 MB1 mRNA (376-394 nt) | Ex 596 nm |
| | 5′-Texas red-TGGAGTTGTCGGTGTAGACTCCA- | Em 615 nm (red) |
| | Dabcyl-3′-(SEQ ID NO: 3) | |
| | Cyclin D 1 MB2 mRNA (698-715 nt) | |
| | 5′-Texas red-CACTTGATCACTCTGGACAAGTG- | |
| | Dabcyl-3′-(SEQ ID NO: 4) | |
| Her-2/neu | Her-2/neu MB1 mRNA (Exon 2) | Ex 346 nm |
| | 5′-Alexa Fluo 350-TAGAGGTGGCGGAGCATCTCTA- | Em 442 nm (Blue) |
| | Dabcyl-3′-(SEQ ID NO: 5) | |
| | Her-2/neu MB2 mRNA (Exon 3) | |
| | 5′-Alexa Fluo 350-CAATCCGCAGCCTCTGCGATTG- | |
| | Dabcyl-3′-(SEQ ID NO:6) | |

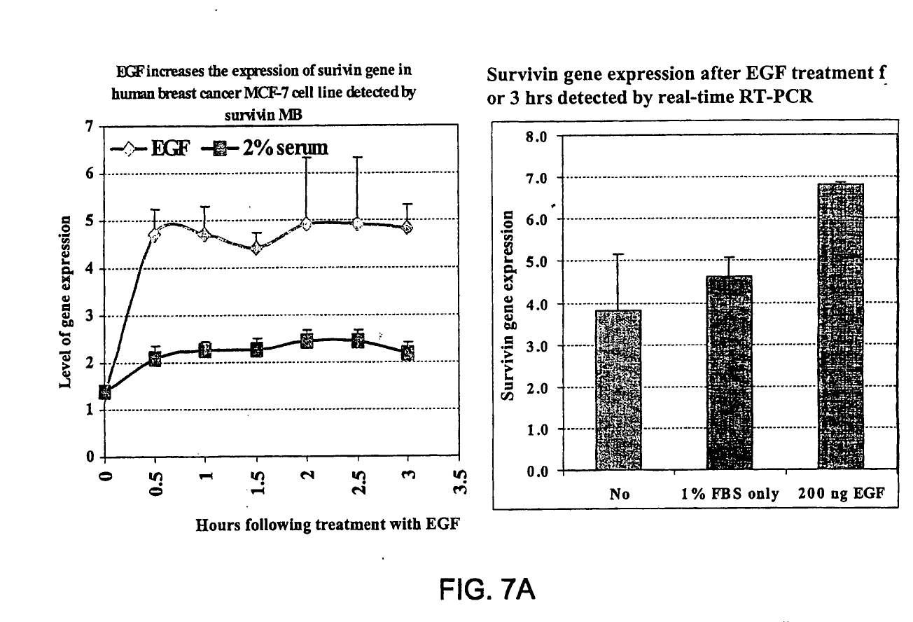

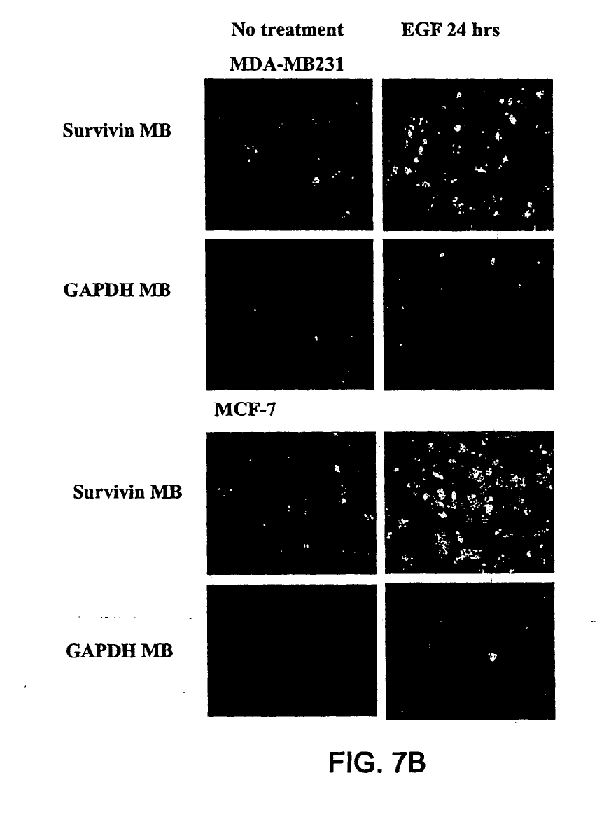

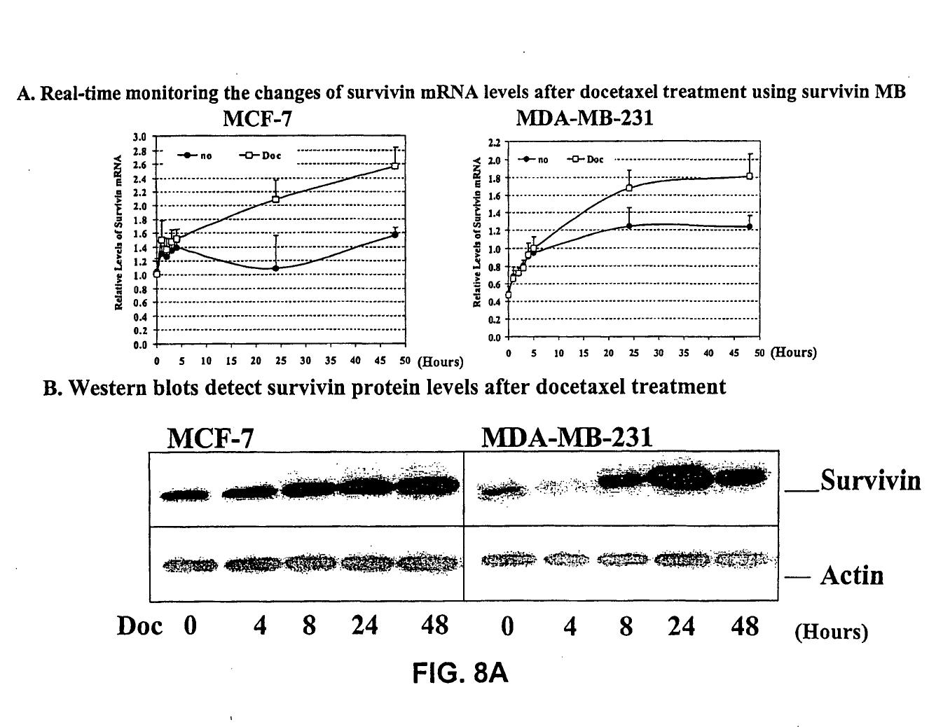

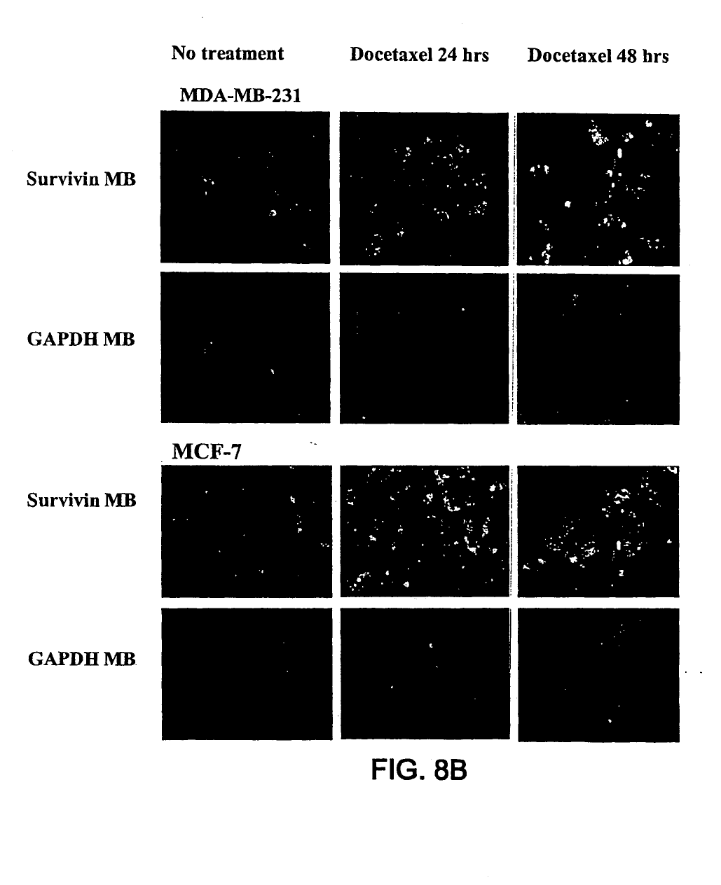

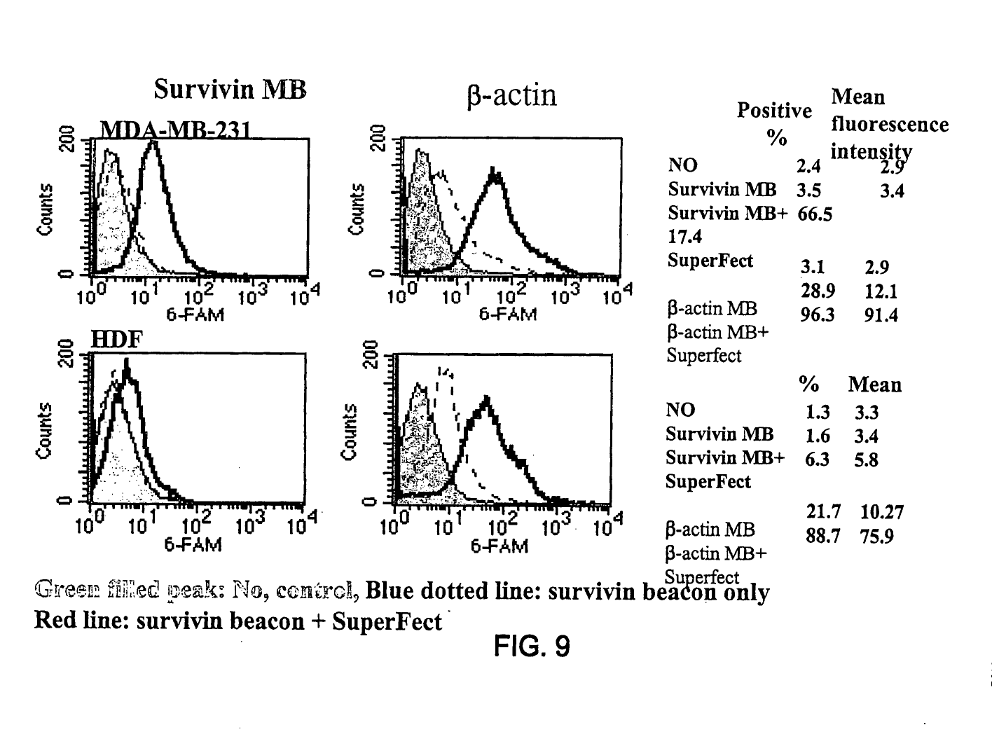

For detection of breast cancer cells with cyclin D 1 and survivin MBs, a mixture of 100 nM of the cyclin D1 and survivin MBs were incubated with acetone-fixed MDA-MB-231, SKBr3, MDA-MB-435, MCF-7 or MCF-10 A cells for 1 hr. The fluorescence intensity was examined under a fluorescence microscope (FIG. 4A). Levels of survivin and cyclin D1 proteins were examined by Western blot analysis are shown in FIG. 4 B, which indicated a correction between the level of survivin and cyclin D1 mRNA detected by the MBs with the protein levels (FIG. 4B). FIG. 7 shows the detection of gene expression in real time in living cells using molecular beacons following treatment of human breast cell cancer line MCF-7 cells with EGF. FIG. 8 showed that treatment of docetaxel increased the level of survivin gene expression in breast cancer cell lines.

### Example 3

Identification of Survivin Expressing Cells in Primary Breast Cancer Tissues

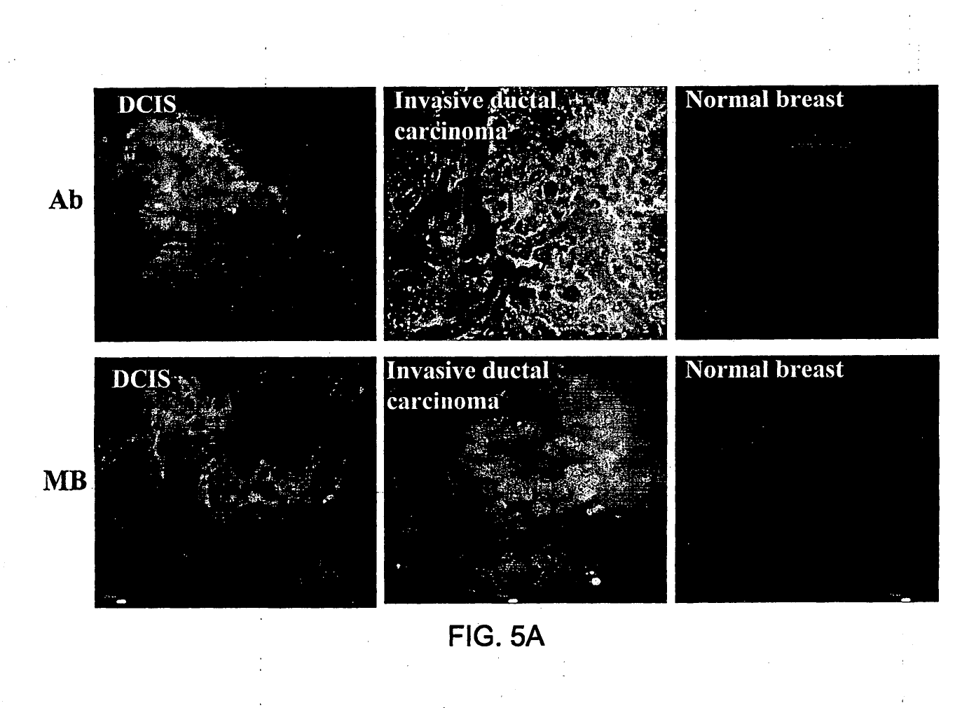

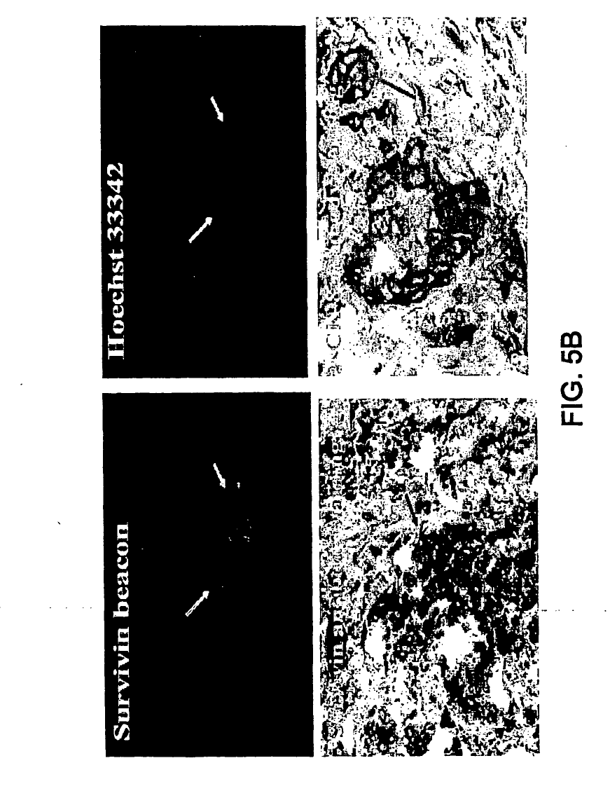

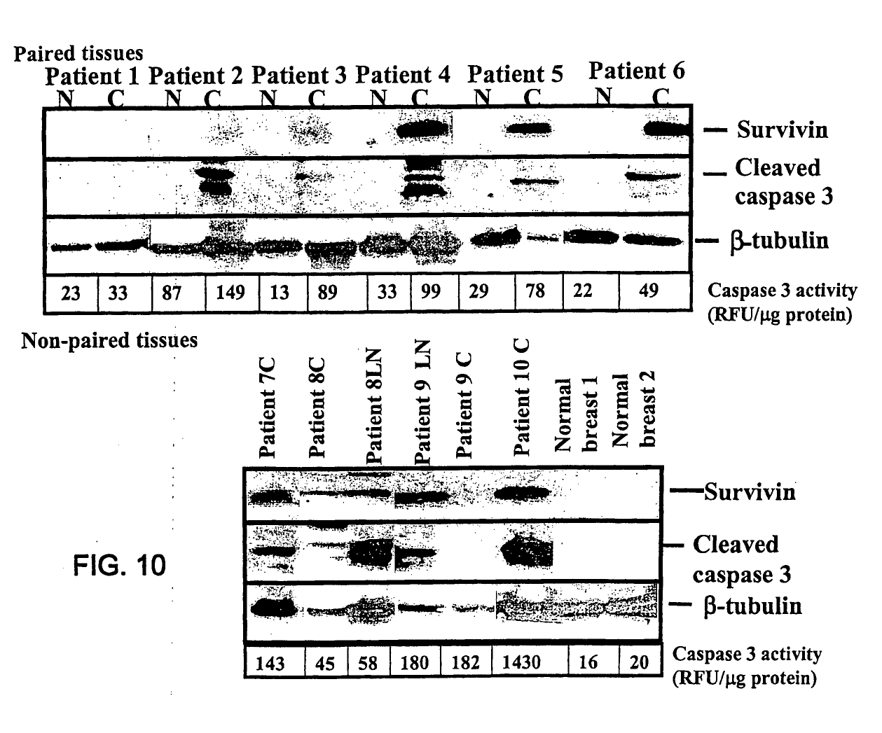

To investigate the feasibility of using survivin as a breast cancer marker, the expression of survivin proteins in paired and non-paired normal and breast cancer tissues by Western blot analysis was examined. It was found that that survivin is expressed in over 73% of breast cancer tissues but not in any of the normal breast tissues FIG. 10). Immunofluorescence staining of frozen tissue sections with survivin antibody also revealed that survivin is highly expressed in invasive ductal carcinoma cells but not in normal breast ductal cells (FIG. 5A). Interestingly, the lesions of DCIS also showed intermediate level of survivin (FIG. 5A). Examination of survivin gene expression on frozen tissue sections using survivin MBs further revealed survivin-expressing cancer cells in DCIS lesion, invasive ductal carcinoma, and metastases in draining lymph node of a breast cancer patient. However, there were no survivin positive cells found in normal breast tissues (FIGS. 5A and B).

### Example 4

Design of MBs for Detection of Mutant K-ras and Survivin mRNA in Pancreatic Cancer Cell and Tissues

Having determined the parameters for beacon structures and hybridization conditions, MBs for detecting the expression of K-ras mutation genes were designed and synthesized. These are depicted in Table 4. Since over 80% of K-ras mutations are found in K-ras codon 12 (Vos et al., J. of Path. 187(3):279-84 (1999)), K-ras MB 1 that detect GGT-GAT (Gly to Asp) transition and K-ras MB2, targeting GGT to GTT mutation, were synthesized (Table 2). GGT to GAT is one of the most common K-ras point mutations in pancreatic cancer (Table 3).

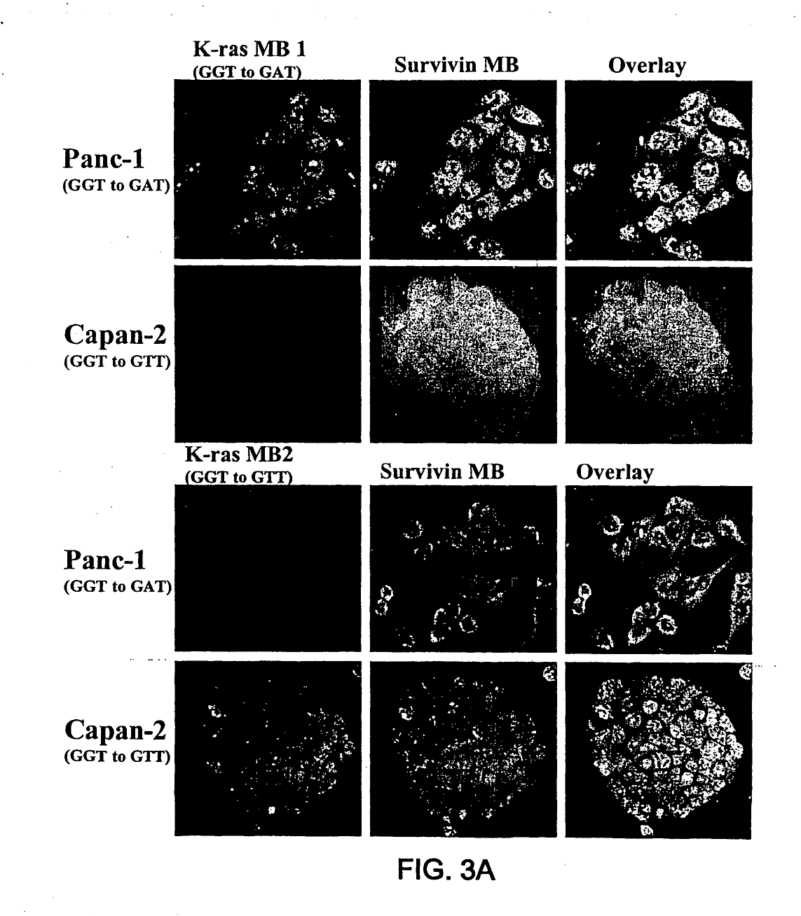

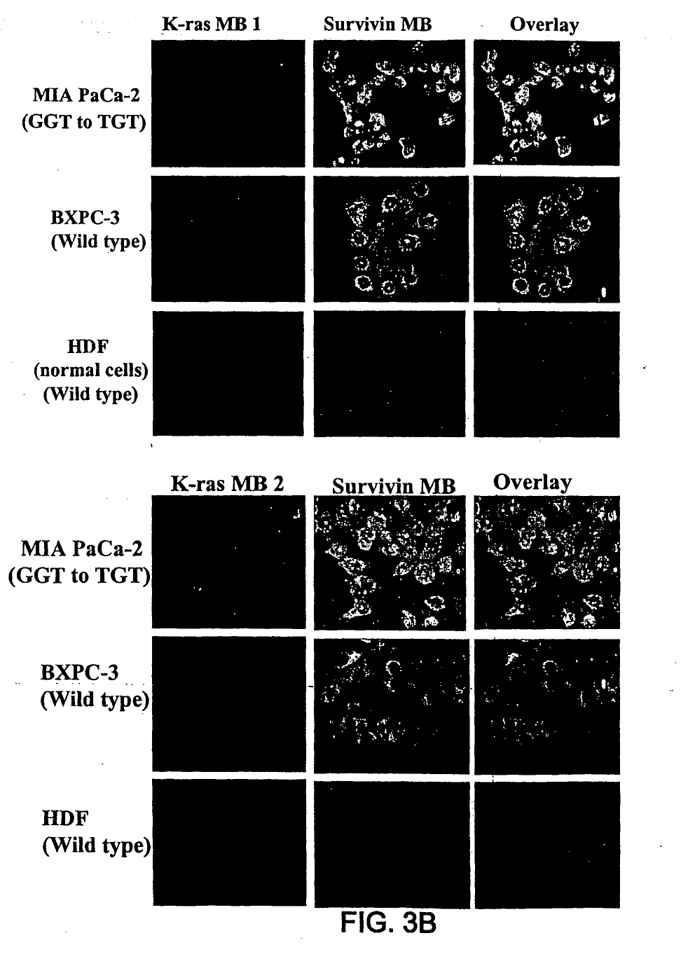

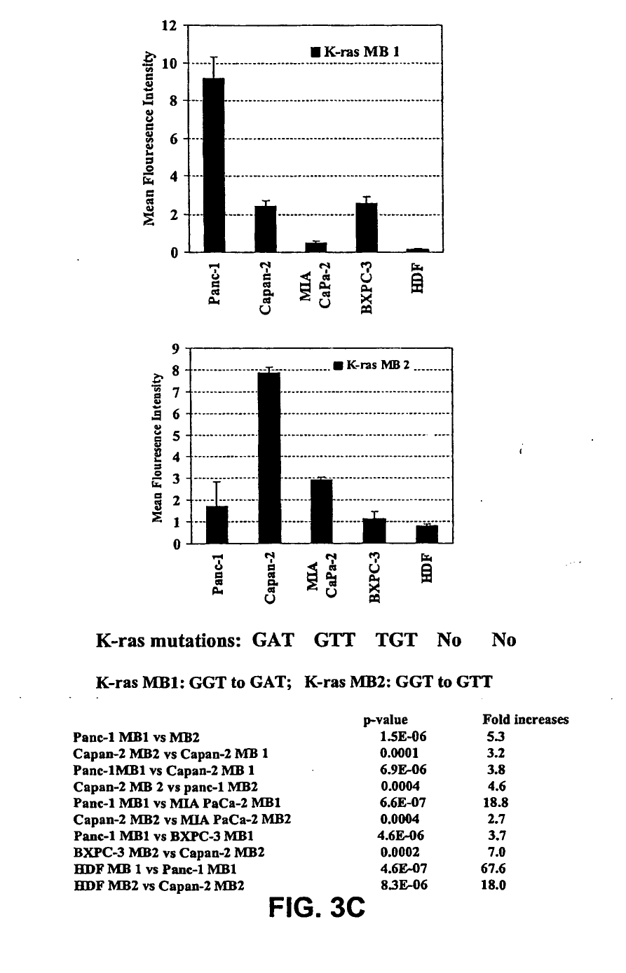

K-ras MB 1 or MB2 selectively binds to its DNA target in vitro and produces stronger fluorescence signal as compared with other non-specific DNA targets (FIG. 2). Examination of specificity of K-ras MBs in pancreatic cancer cell lines also demonstrated that K-ras MBs can detect pancreatic cancer cells with a specific k-ras mutation. As shown in the FIG. 3, Panc-1 cell line has a K-ras GGT to GAT mutation and showed strong fluorescence intensity after incubating with K-ras MB 1, but displayed a weak fluorescence in K-ras MB2-stained cells. Capan-2 cell line contains a K-ras GGT to GTT mutation and a brighter fluorescence was detected in K-ras MB 2 stained cells. Both cell lines expressed a high level of survivin as detected by survivin MB (FIG. 3A). On the other hand, K-ras MBs did not produce strong fluorescence signaling in MIA PaCa-2 cell line, which has a K-ras GGT to TGT mutation, or K-ras wild type BXPC-3 cells. However, those cells were positive for survivin MB staining. Importantly, incubation of K-ras and survivin MBs did not produce detectable fluorescence signaling in a normal cell line (HDF), which is generated from normal dermal fibroblasts (FIG. 3B).

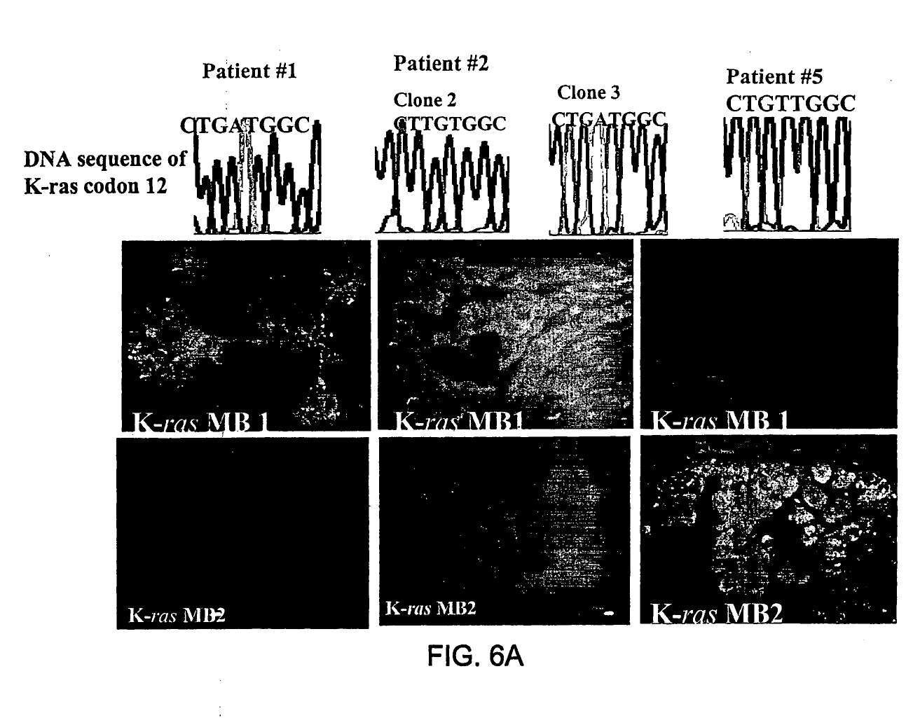

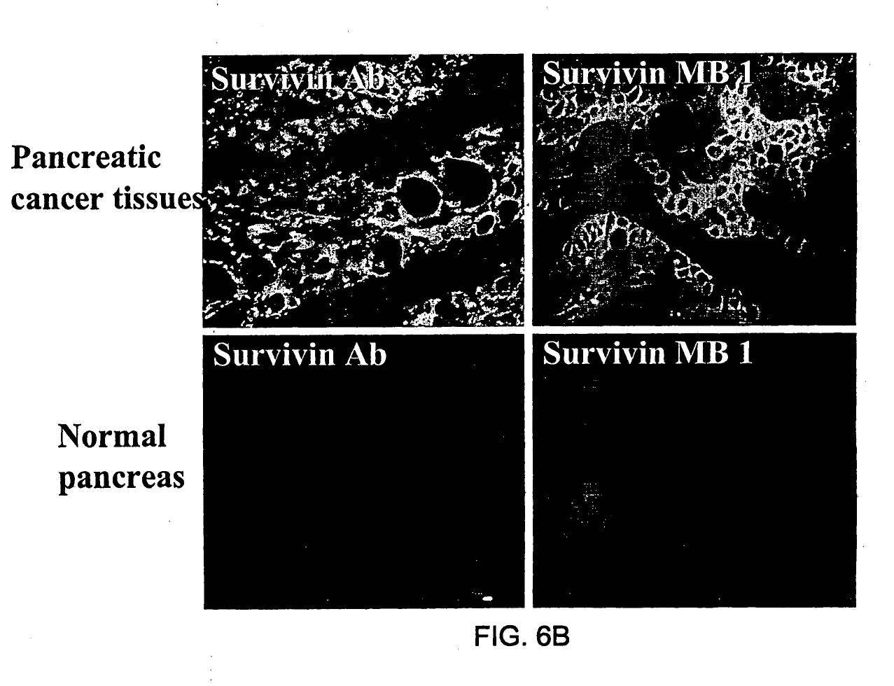

K-ras MBs were also able to detect pancreatic cancer cells with K-ras mutations on Frozen tissue sections. After incubating with K-ras MB 1 or K-ras MB 2 for 1 hour and counterstained with Hoechst 33342 (blue), The slides were observed under fluorescence microscope with a digital imaging system. K-ras MB 1 detected the cancer cells expressing a GGT to GAT mutant K-ras gene on the frozen sections of pancreatic cancer tissues from patient #1 and #2, which contained K-ras codon 12 GGT to GAT mutation. However, bright red fluorescent cells were found on frozen sections of pancreatic cancer tissues from patient #5, which had a K-ras GGT to GTT mutation after incubation with K-ras MB 2 but not with K-ras MB1 (FIG. 6A).

The feasibility of detection of survivin expression in pancreatic cancer tissues by survivin MB was also examined. At first, expression of survivin in the pancreatic cancer tissues was demonstrated by immunofluorescence staining with an anti-survivin monoclonal antibody. Inventors showed that survivin protein was highly expressed in pancreatic cancer tissues but was undetectable in normal pancreas (FIG. 6B).

TABLE 2
Design of molecular beacons for K-ras codon 12 mutation and
survivin genes and corresponding target sequences
A. Design of the MBs for detecting mutant K-ras and survivin mRNAs

| MBs | Target sequences | Design of the MBs |
|---|---|---|
| K-ras MB 1 | K-ras codon 12 GGT | 5′-Cy3-CCTACGCCATCAGCTCCGTAGG-Dabcyl-3′ |
| | to GAT mutation | (SEQ ID NO 7) |
| K-ras MB 2 | K-ras codon 12 GGT | 5′-Texas-red-CCTACGCCAACAGCTCCGTAGG-Dabcyl-3′ |
| | to GTT mutation | (SEQ ID NO 8) |
| Survivin MB 1 | Survivin cDNA from | 5′-Cy3 (Alexia-Fluo488 or 6-FAM)- |
| | 27 to 43 nucleotide | CTGAGAAAGGGCTGCCAGTCTCAG-Dabcyl-3′ |
| | | (SEQ ID NO 2) |
| Survivin MB 2 | Survivin cDNA from | 5′FITC-TGGTCCTTGAGAAAGGGCGACCA-Dabcyl-3′ |
| | 32 to 51 nucleotide | (SEQ ID NO 9) |
| GAPDH MB | GAPDH cDNA from | 5′-Cy3-GAGTCCTTCCACGATACCGACTC-Dabcyl-3′ |
| | 504 to 521 nucleotide | (SEQ ID NO 10) |

B. Oligonucleotides served as DNA targets for K-ras and survivin MBs

| DNA Targets | Synthesized Oligonucleotide sequences |
|---|---|
| K-ras wild type | 5′-GTA GTT GGA GCT GGT GGC GTA GGC AAG AGTGCCTTGACGATACAGCTAATT CAG-3′ |
| (GGT) | |
| K-ras Mut 1 (GAT) | 5′-GTA GTT GGA GCT GAT GGC GTA GGC AAG AGTGCCTTGACGATACAGCTAATT CAG-3′ |
| K-ras Mut 2 (GTT) | 5′-GTA GTT GGA GCT GTT GGC GTA GGC AAGAGTGCCTTGACGATACAGCTAATT CAG-3′ |
| Survivin | 5′-CCTGCCTGGCAGCCCTTTCTCAAGGACCACCGCATCTCTACATTCAAGAAC-3′ |

TABLE 3
K-ras mutations in pancreatic cancer cell lines

| | | | | Percentage of |
|---|---|---|---|---|
| | | | | the Mutation |
| | | | | in Primary |
| | K-ras | | Predicated | Pancreatic |
| Cell lines | mutation | Alteration | Products | Tissues |
| Panc-1 | Yes, | GGT to GAT | GLY to ASP | 48+/101 (48%) |
| | Codon 12 | | | |
| Capan-2 | Yes, | GGT to GTT | GLY to VAL | 33+/101 (33%) |
| | Codon 12 | | | |
| Miapaca-2 | Yes, | GGT to TGT | GLY to CYS | 2+/101 (2%) |
| | Codon 12 | | | |
| PSN-1 | Yes, | GCT to CGT | GLY to ARG | 9+/101 (9%) |
| | Codon 12 | | | |
| BXPC-3 | No | No | GLY | No |
| HDF | No | No | GLY | No |

TABLE 4

Design of molecular beacons targeting K-ras
codon 12 mutations

5′-ATG ACT GAA TAT AAA CTT GTG GTA GTT GGA GCT GGT
GGC GTA GGC AAG AGT GCC TTG ACG-3′

| | |
|---|---|
| Molecular Beacons | |
| GGT-GAT: | |
| 5′-Cy 3- | (SEQ ID NO: 7) |
| CCTACGCCATCAGCTCCGTAGG-Dabcyl-3′- | |
| GGT-GTT: | |
| 5′-Cy-3- | (SEQ ID NO: 8) |
| CCTACGCCAACAGCTCCGTAGG-Dabcyl-3′- | |
| GGT-CGT: | |
| 5′-Cy-3- | (SEQ ID NO: 11) |
| CCTACGCCACGAGCTCCGTAGG-Dabcyl-3′- | |
| GGT-TGT: | |
| 5′-Cy-3- | (SEQ ID NO: 12) |
| CCTACGCCACAAGCTCCGTAGG-Dabcyl-3′- | |
| GGT-GCT: | |
| 5′-Cy-3- | (SEQ ID NO: 13) |
| CCTACGCCAGCAGCTCCGTAGG-Dabcyl-3′- | |

Codon 12 = underlined

The above specification, examples, and data provide a complete description of the manufacture and use of the invention. Unless otherwise specified, all patent and non-patent references cited are hereby incorporated by reference for background information only. Since many embodiments of the invention can be made without departing from the spirit and scope of the invention, the invention resides in the claims hereinafter appended.

### LIST OF REFERENCES

1. Altieri D C and Marchisio P C. Survivin apoptosis: an interloper between cell death and cell proliferation in cancer. Lab. Invest. 79:1327-33, 1999.
2. Altieri D C. The molecular basis and potential role of survivin in cancer diagnosis and therapy. Trends in Mol. Med. 7:542-547, 2001.
3. Ambrosini G, Adida C, Altieri D C, A novel anti-apoptosis gene, survivin, expressed in cancer and lymphoma. Nature Medicine 3:917-21, 1997.
4. Anker P, Lefort F, Vasioukhin V, Lyautey J, Lederrey C, Chen X Q, Stroun M, Mulcatl H E, and Farthing M J G. K-ras mutations are found in DNA extracted from the plasma c patients with colorectal cancer. Gastroenterology 112:1114-1120, 1997.
5. Asanuma K, Moriai R, Yajima T, Yagihashi A, Yamada M, Kobayashi D and Watanab N. Survivin as a radioresistance factor in pancreatic cancer. Japanese Journal of Cancer Research 91:1204-9, 2000.
6. Azuhata T, Scott D, Takamizawa S, Wen J, Davidoff A, Fukuzawa M, Sandler A. The inhibitor of apoptosis protein survivin is associated with high-risk behavior of neuroblastoma. Journal of Pediatric Surgery 36:1785-91, 2001.
7. Barkin J S and Goldstein J A. Diagnostic approach to pancreatic cancer. Gastroenterology Clinics of North America 28:709-22, 1999.
8. Berrozpe G, Schaeffer J, Peinado M A, Real F X and Perucho M. Comparative analysis of mutations in the p53 and K-ras genes in pancreatic cancers. International Journal of Cancer, 58:185-191, 1994.
9. Buchler P, Conejo-Garcia J R, Lehmann G, Muller M, Emrich T, Reber H A, Buchler M W and Friess H. Real-time quantitative PCR of telomerase mRNA is useful for the differentiation of benign and malignant pancreatic disorders. Pancreas 22:331-40, 2001
10. Clayton S J, Scott F M, Walker J, Callaghan K, Haque K, Liloglou T, Xinarianos G, Shawcross S, Ceuppens P, Field J K and Fox J C. K-ras point mutation detection in lung cancer: comparison of two approaches to somatic mutation detection using ARMS allele-specific amplification. Clinical Chemistry 46:1929-38, 2000.
11. Deveraux Q L. Roy N. Stennicke H R. Van Arsdale T. Zhou Q. Srinivasula S M. Alnemri E S. Salvesen G S. Reed J C. IAPs block apoptotic events induced by caspase-8 and cytochrome c by direct inhibition of distinct caspases. EMBO Journal. 17(8):2215-23, 1998.
12. Dirks R W. Molenaar C. Tanke H J. Methods for visualizing RNA processing and transport pathway in living cells. Histochem. & Cell Biol. 115(1): 3-11, 2001.
13. Dubertret B, Calame M, and Libchaber A. Single-mismatch detection using gold-quenched fluorescent oligonucleotides. Nat Biotechnol 19:365-370, 2001.
14. Fischer C, Buthe J, Nollau P, Hollerbach S, Schulmann K, Schmiegel W, Wagener C and Tschentscher P. Enrichment of mutant KRAS alleles in pancreatic juice by subtractive iterative polymerase chain reaction. Laboratory Investigation 81:827-31, 2001.
15. Futakawa N, Kimura W, Yamagata S, Zhao B, Ilsoo H, Inoue T, Sata N, Kawaguchi Y, Kubota Y and Muto T. Significance of K-ras mutation and CEA level in pancreatic juice in the diagnosis of pancreatic cancer. Journal of Hepato-Biliary-Pancreatic Surgery 7:63-71, 2000.
16. Goggins M, Hruban R H and Kern S E. BRCA2 is inactivated late in the development c pancreatic intraepithelial neoplasia: evidence and implications. American Journal of Pathology 156:1767-71, 2000.
17. Gold E I, Epidemiology of and risk factors for pancreatic cancer. Surg. Clin. North Am. 75:819-839, 1995.
18. Harris J R, Morrow M, Norton L: Malignant tumors of breast. In Detita VT, Hellman S, Rosenberg S A (eds): “Cancer: Principles and practice of oncology.” Philadelphia: Lippincott-Raven; 1997. P. 1557-1616.
19. Hosch S, Braun S, and Pantel K. Characterization of disseminated tumor cells. Seminar in Surgerical Oncology, 20:265-271, 2001.
20. Hruban R H, Stum P D, Slebos R J, Wilentz R E, Musler A R, Yeo C J, Sohn T A, Velthuy and Offerhaus G J A. Can K-ras codon 12 mutations be used to distinguish bile duct proliferations from metastases in the liver. American Journal of Pathology 151:943-94
21. Janocko L E. Brown K A. Smith C A. Gu L P. Pollice A A. Singh S G. Julian T. Wolmark N. Sweeney L. Silverman J F. Shackney S E. Distinctive patterns of Her-2/neu, c-myc, and cyclin D1 gene amplification by fluorescence in situ hybridization in primary human breast cancers. Cytometry. 46(3): 136-49, 2001.
22. Kappler M, Kohler T, Kampf C, Diestelkotter P, Wurl P, Schmitz M, Bartel F, Lautenschlager C, Rieber E P, Schmidt H, Bache M, Taubert H, Meye A. Increased survivin transcript levels: an independent negative predictor of survival in soft tissue sarcoma patients. International Journal of Cancer. 95:360-3, 2001.
23. Kato J, Kuwabara Y, Mitani M, Shinoda N, Sato A, Toyama T, Mitsui A, Nishiwaki T Moriyama S, Kudo J and Fujii Y. Expression of survivin in esophageal cancer: correlation with the prognosis and response to chemotherapy. International Journal of Cancer 95:92-5, 2001.
24. LaCasse E C, Baird S, Korneluk R G and MacKenzie A E. The inhibitors of apoptosis (IAPs) and their emerging role in cancer. Oncogene 17:3247-59, 1998.
25. Lakowicz J R. Principles of Fluorescence Spectroscopy, 2^{nd} ed, Kluwer Academic/Plenum Publishers, New York, N.Y., 1999.
26. Li F, Ackermann E J, Bennett C F, Rothermel A L, Plescia J, Tognin S, Villa A, Marchisio P C, and Altieri D C. Pleiotropic cell-division defects and apoptosis induced by interference with survivin function. Nature cell Biology 1:461-466, 1999.
27. Li F, Ambrosini G, Chu E Y, Plescia J, Tognin S, Marchisio P C and Altieri D C. Control of apoptosis and mitotic spindle checkpoint by survivin. Nature 396:580-4, 1998.
28. Luttges J, Schlehe B, Menke M A, Vogel I, Henne-Bruns D and Kloppel G. The K-ras mutation pattern in pancreatic ductal adenocarcinoma usually is identical to that in associated normal, hyperplastic, and metaplastic ductal epithelium. Cancer 85:1703-10, 1999.
29. Minamoto T, Mai M and Ronai Z. K-ras mutation: early detection in molecular diagnosis and risk assessment of colorectal, pancreas, and lung cancers—a review. Cancer Detection & Prevention 24:1-12, 2000.
30. Moore M. Urine detection of survivin and diagnosis of bladder cancer. Journal of insurance medicine 33:202-3, 2001.
31. Myung S J, Kim M H, Kim Y S, Kim H J, Park E T, Yoo K S, Lim B C, Wan Seo D, Lee S K, Min Y I and Kim J Y. Telomerase activity in pure pancreatic juice for the diagnosis of pancreatic cancer may be complementary to K-ras mutation. Gastrointestinal Endoscopy 51:708-13, 2000.
32. Nakaizumi A, Uehara H, Takenaka A, Uedo N, Sakai N, Yano H, Ohigashi H, Ishikawa O, Ishiguro S, Sugano K and Tatsuta M. Diagnosis of pancreatic cancer by cytology an measurement of oncogene and tumor markers in pure pancreatic juice aspirated by endoscopy. Hepato-Gastroenterology 46:31-7, 1999.
33. National Institute of Health: Nationa Cancer Institute SEER Cancer Statistics Review 1973-1990, NIH publication 93-2789. Bethesda, Md. 1993.
34. Nomoto S, Nakao A, Ando N, Takeda S, Kasai Y Inoue S, Kaneko T and Takagi H. Clinical application of K-ras oncogene mutations in pancreatic carcinoma: detection of micrometastases. Seminars in Surgical Oncology 15:4046, 1998.
35. Okai T, Watanabe H, Yamaguchi Y, Mouri I, Motoo Y and Sawabu N. EUS and K-ras analysis of pure pancreatic juice collected via a duodenoscope after secretin stimulation for diagnosis of pancreatic mass lesion: a prospective study. Gastrointestinal Endoscopy 50:797-803, 1999.
36. O'Shaughnessy J A. Ljung B M. Dooley W C. Chang J. Kuerer H M. Hung D T. Grant M D. Khan S A. Phillips R F. Duvall K. Euhus D M. King B L. Anderson B O. Troyan S L. Kim J. Veronesi U. Cazzaniga M. Ductal lavage and the clinical management of women at high risk for breast carcinoma: a commentary. Cancer. 94(2):292-8, 2002.
37. Parker S L, Tong T and Bolden S. Cancer statistics. CA. Cancer J. Clin. 46:5-27, 1996.
38. Pervin S. Singh R., and Chaudhuri G. Nitric oxide-induced cytostasis and cell cycle arrest of a human breast cancer cell line (MDA-MB-231): Potential role of cyclin D 1. Proc. Natl. Acad. Sci. USA, Vol. 98, Issue 6, 3583-3588, 2001.
39. Poddar S K. Symmetric vs asymmetric PCR and molecular beacon prob in the detection of a target gene of adenovirus. Molecular & Cellular Prodes. 14:25-32, 2000.
40. Poller D N. Galea M. Pearson D. Bell J. Gulick W J. Elston C W. Blarney R W. Ellis I O. Nuclear and flow cytometric characteristics associated with overexpression of the c-erbB-2 oncoprotein in breast carcinoma. Breast Cancer Res. & Treat. 20(1):3-10, 1991.
41. Puig P. Urgell E. Capella G. Sancho F J. Pujol J. Boadas J. Farre A. Lluis F. Gonzalez-Sastre F. Mora J. A highly sensitive method for K-ras mutation detection is useful in diagnosis of gastrointestinal cancer. International Journal of Cancer. 85(1):73 7, 2000.
42. Queneau P E, Adessi G L, Thibault P, Cleau D, Heyd B, Mantion G and Carayon P. Early detection of pancreatic cancer in patients with chronic pancreatitis: diagnostic utility of a K-ras point mutation in the pancreatic juice. American Journal of Gastroenterology 96:700-4, 2001.
43. Ramachandra S. Machin L. Ashley S. Monaghan P. Gusterson B A. Immunohistochemical distribution of c-erbB-2 in in situ breast carcinoma—a detailed morphological analysis. J. of Path. 161(1):7-14, 1990.
44. Rozenblum E, Schutte M, Goggins M, Hahn S A, Panzer S, Zahurak M, Goodman S N, Sohn T A, Hruban R H, Yeo C J and Kern S E. Tumor-suppressive pathways in pancreatic carcinoma, Cancer Research 57: 1731-1734, 1997.
45. Sarela A L, Macadam R C A, Farmery S M, Markham A F and Guillou P J. Expression of the anti-apoptosis gene, survivin, predicts death from recurrent colorectal carcinoma. Gut 46(5):645-650, 2000.
46. Saton K, Kaneko K, Hirota M, Masamune A, Satoh A and Shimosegawa. Expression o survivin is correlated with cancer cell apoptosis and is involved in the development of human pancreatic duct cell tumors. Cancer 92:271-278, 2001.
47. Seki K, Suda T, Aoyagi Y, Sugawara S, Natsui M, Motoyama H, Shirai Y, Sekine T, Kawai H, Mita Y, Waguri N, Kuroiwa T, Igarashi M and Asakura H. Diagnosis of pancreatic adenocarcinoma by detection of human telomerase reverse transcriptase messenger RNA in pancreatic juice with sample qualification. Clinical Cancer Research 7:1976-81, 2001.
48. Seregni E, Ferrari L, Martinetti A and Bombardieri E. Diagnostic and prognostic tumor markers in the gastrointestinal tract. Seminars in Surgical Oncology 20:147-166, 2001.
49. Shibata K, Mori M, Kitano S and Akiyoshi T. Detection of ras gene mutations in peripheral blood of carcinoma patients using CD45 immunomagnetic separation and nested mutant allele specific amplification. International Journal of Oncology 12:1333-8, 1998.
50. Sokol D L, Zhang X, Lu P, and Gewirtz A M. Real time detection of DNA.RNA hybridization in living cells. Proc Natl Acad Sci USA 95:11538-11543, 1998.
51. Swana H S, Grossman D, Anthony J N, Weiss R M, Altieri D C. Tumor content of the antiapoptosis molecule survivin and recurrence of bladder cancer. New England Journal of Medicine. 341:452-3, 1999.
52. Tamm I, Wang Y, Sausville E, Scudiero D A, Vigna N, Oltersdorf T and Reed J C. IAP-family protein survivin inhibits caspase activity and apoptosis induced by Fas (CD95), Bax, caspases, and anticancer drugs. Cancer Research 58:5315-20, 1998.
53. Tanaka K, Iwamoto S, Gon G, Nohara T, Iwamoto M and Tanigawa N. Expression of survivin and its relationship to loss of apoptosis in breast carcinomas. Clin. Cancer Res. 6:127-34, 2000.
54. Tascilar M, Caspers E, Sturm P D, Goggins M, Hruban R H and Offerhaus G J. Role of tumor markers and mutations in cells and pancreatic juice in the diagnosis of pancreatic cancer. Annals of Oncology 10 Suppl 4:107-10, 1999.
55. Tsourkas A, Behlke M, Rose S and Bao G. Hybridization kinetics and thermodynamics molecular beacons with structural variations. Submitted to Biophysical J, 2002.
56. Tyagi S and Kramer F R. Molecular beacons: probes that fluoresce upon hybridization. Nature Biotechnol 14, 303-308, 1996.
57. Tyagi S, Bratu D P, and Kramer F R. Multicolor molecular beacons for allele discrimination. Nat Biotechnol 16:49-53, 1998.
58. Urgell E, Puig P, Boadas J, Capella G, Queralto J M, Boluda R, Antonijuan A, Farre A, Lluis F, Gonzalez-Sastre F and Mora J. Prospective evaluation of the contribution of K ras mutational analysis and CA 19-9 measurement to cytological diagnosis in patients with clinical suspicion of pancreatic cancer. European Journal of Cancer 36:2069-75, 2000.
59. Velculescu V E, Madden S L, Zhang L, Lash A E, Yu J, Rago C, Lal A, Wang C J, Beaudry G A, Ciriello K M, Cook B P, Dufault M R, Ferguson A T, Gao Y, He T C, Hermeking H, Hiraldo S K, Hwang P M, Lopez M A, Luderer H F, Mathews B, Petroziello J M, Polyak K, Zawel L Kinzler K W, et al. Analysis of human transcriptomes [letter]. Nature Genetics 23:387-8, 1999.
60. Vogel I, Kalthoff H, Disseminated tumour cells, their detection and significance for prognosis of gastrointestinal and pancreatic carcinomas. Virchows Archiv 439:109-17, 2001.
61. Vogel I, Kruger U, Marxsen J, Soeth E, Kalthoff H, Henne-Bruns D, Kremer B, Juhl H Disseminated tumor cells in pancreatic cancer patients detected by immunocytology: a new prognostic factor. Clinical Cancer Research 5:593-9, 1999.
62. Vos C B. Ter Haar N T. Peterse J L. Cornelisse C J. van de Vijver M J. Cyclin D1 gene amplification and overexpression are present in ductal carcinoma in situ of the breast. J. of Path. 187(3):279-84, 1999 Feb.
63. Watanabe H, Yamaguchi Y, Ha A, Hu Y X, Motoo Y, Okai T, Yoshimura T and Sawabu N. Quantitative determination of K-ras mutations in pancreatic juice for diagnosis of pancreatic cancer using hybridization protection assay. Pancreas 17:341-7, 1998.
64. Weinstat-Saslow D, Merino M J, Manrow R E, Lawrence J A, Bluth R F, Wittenbel K D, Simpson J F, Page D L, Steeg P S. Overexpression of cyclin D mRNA distinguishes invasive and in situ breast carcinomas from non-malignant lesions. Nat Med 1995 December;1(12):1257-60.
65. Yamada H, Yoshida T, Sakamoto H, Terada M and Sugimura T. Establishment of a human pancreatic adenocarcinoma cell line (PSN-1) with amplifications of both c-myc and activated c-Ki-ras by a point mutation. Biochemical & Biophysical Research Communications, 140:167-73, 1986.
66. Yang L, and Zhang Y. Differential regulation of apoptotic cell death pathway in tumor cells affects GCV sensitivity in HSV-TK modified human tumor cell lines. Fourth Annual American Society for Gene Therapy, Seattle, May 30 to Jun. 3, 2001.
67. Z'graggen K, Centeno B A, Castillo F, Jimenez R E, Werner J, and Warshaw A L. Biological implication of tumor cells in blood and bone marrow of pancreatic cancer patients, Surgery 129:537-545, 2001.

We claim:

1. A method of detecting the presence of at least one tumor marker mRNA in a sample comprising:
i) providing a sample of cells for analysis;
ii) treating the sample with an oligonucleotide that targets the tumor marker mRNA, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the target sequence under suitable hybridization conditions, wherein the presence, absence or amount of target sequence present in the sample is correlated with a change in detectable signal associated with at least one donor or acceptor moiety of the oligonucleotide; and
iv) detecting, identifying or quantitating the presence of a tumor marker based upon the presence, absence or amount of the hybridization of the oligonucleotide to the target sequence that is determined.

2. The method of claim 1, wherein the tumor marker is one or more of the markers selected from the group consisting of survivin, cyclin D1, Her2/neu, a mutant K-ras, chymotrypsinogen, basic fiborblast growth factor, carcinoembryonic antigen, prostate, specific antigen, alpha-fetalprotein, beta-2-microglobulin, bladder tumor antigen, chromogranin A, neuron-specific enolase, S-100, TA-90, tissue polypeptide antigen and human chorionic gonadotropin

3. The method of claim 1, wherein the sample taken from at least one source selected from the group consisting of blood, urine, pancreatic juice, ascites, breast ductal lavage, nipple aspiration, needle biopsy or tissue.

4. The method of claim 3, wherein the tissue is a biopsy from the pancreas or breast.

5. The method of claim 3, wherein the tissue is a frozen section.

6. The method of claim 1, wherein the sample is taken from a breast ductal lavage.

7. The method of claim 1, wherein the sample is taken from pancreatic juice.

8. The method of claim 1, wherein the quantification of the presence of the tumor marker is accomplished by FACS-scan analysis.

9. The method of claim 1, wherein the olignucleotide is one or more selected from the group consisting of SEQ ID NOS:1, 2, 3, 4, 5, 6, 7, 8, 9, 10, 11, 12 and 13.

10. The method of claim 1, wherein the oligonucleotide targets the tumor marker survivin.

11. The method of claim 10, wherein the oligonucleotide is one or more selected from the group consisting of SEQ ID NOS: 1, 2 and 9.

12. The method of claim 1, wherein the oligonucleotide targets the tumor marker cyclin D1.

13. The method of claim 12, wherein the oligonucleotide is one or more selected from the group consisting of SEQ ID NOS: 3 and 4.

14. The method of claim 1, wherein the oligonucleotide targets the tumor marker Her2/neu.

15. The method of claim 14, wherein the oligonucleotide is one or more selected from the group consisting of SEQ ID NOS: 5 and 6.

16. The method of claim 1, wherein the oligonucleotide targets the tumor marker a K-ras mutant gene.

17. The method of claim 16, wherein the oligonucleotide is one or more selected from the group consisting of SEQ ID NOS: 7, 8, 11, 12 and 13.

18. A method of detecting the presence of a mutant gene in a tumor cell comprising:
i) providing a sample of tumor cells for analysis;
ii) treating the sample with an oligonucleotide that targets the mutant gene, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the oligonucleotide to the mutant gene target sequence under suitable hybridization conditions, wherein the presence, absence or amount of mutant gene target sequence present in the sample is correlated with a change in detectable signal associated with at least one donor or acceptor moiety of the oligonucleotide; and
iv) detecting, identifying or quantitating the presence of a mutant gene based upon the presence, absence or amount of the hybridization of the oligonucleotide to the mutant gene target sequence that is determined.

19. The method of claim 18, wherein the mutant gene is a mutant K-ras gene.

20. The method of claim 19, wherein the quantification of the presence of the mutant gene is accomplished by FACS-scan analysis.

21. The method of claim 19, wherein the oligonucleotide is one or more selected from the group consisting of SEQ ID NOS: 7, 8, 11, 12, and 13.

22. A method of monitoring alterations in gene expression in viable cells comprising:
i) providing a sample of viable cells for analysis;
ii) treating the sample with an oligonucleotide that targets a particular gene sequence, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the target sequence under suitable hybridization conditions, wherein the presence, absence or amount of target sequence present in the sample is correlated with a change in detectable signal associated with at least one donor or acceptor moiety of the oligonucleotide; and
iv) detecting, identifying or quantitating the alteration in the expression of the particular gene based upon the presence, absence or amount of the hybridization of the oligonucleotide to the target sequence that is determined.

23. The method of claim 22, wherein the quantification of the alteration in gene expression is accomplished by FACScan analysis and by a fluorescence microplate reader.

24. A method of detecting or monitoring presence or progression of breast cancer in a subject comprising:
i) providing a sample of cells from said subject for analysis;
ii) treating the sample with an oligonucleotide that targets a breast cancer marker gene sequence, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the target oligonucleotide sequence to the breast cancer marker gene sequence under suitable hybridization conditions, wherein the presence, absence or amount of target oligonucleotide sequence present in the sample is correlated with a change in detectable signal associated with at least one donor or acceptor moiety of the oligonucleotide; and
iv) detecting, identifying or quantitating the presence or progression of breast cancer based upon the presence, absence or amount of the hybridization of the oligonucleotide to the breast cancer target sequence that is determined.

25. The method of claim 24, wherein the breast cancer marker is one or more of the markers selected from the group consisting of survivin, cyclin D1, Her2/neu, basic fiborblast growth factor, EGF receptor and carcinoembryonic antigen.

26. The method of claim 24, wherein the sample is taken from at least one source selected from the group consisting of blood, urine, breast ductal lavage, ascites, nipple aspiration, needle biopsy or tissue.

27. The method of claim 26, wherein the tissue is a biopsy from a breast or lymph node.

28. The method of claim 26, wherein the tissue is a frozen section.

29. The method of claim 24, wherein the quantification of the presence or progression of the breast cancer is accomplished by FACS-scan analysis.

30. The method of claim 24, wherein the olignucleotide targets the breast cancer marker survivin.

31. The method of claim 30, wherein the oligonucleotide is one or more selected from the group consisting of SEQ ID NOS: 1, 2 and 9.

32. The method of claim 24, wherein the oligonucleotide targets the breast cancer marker cyclin D1.

33. The method of claim 32, wherein the oligonucleotide is one or more selected from the group consisting of SEQ ID NOS: 3 and 4.

34. The method of claim 24, wherein the oligonucleotide targets the breast cancer marker Her2/neu.

35. The method of claim 34, wherein the oligonucleotide is one or more selected from the group consisting of SEQ ID NOS: 5 and 6.

36. A method of detecting or monitoring presence or progression of pancreatic cancer in a subject comprising:
i) providing a sample of cells from said subject for analysis;
ii) treating the sample with an oligonucleotide that targets a pancreatic cancer marker gene sequence, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the target oligonucleotide sequence to the pancreatic cancer marker gene sequence under suitable hybridization conditions, wherein the presence, absence or amount of target oligonucleotide sequence present in the sample is correlated with a change in detectable signal associated with at least one donor or acceptor moiety of the oligonucleotide; and
iv) detecting, identifying or quantitating the presence or progression of pancreatic cancer based upon the presence, absence or amount of the hybridization of the oligonucleotide to the breast cancer target sequence that is determined.

37. The method of claim 36, wherein the pancreatic cancer marker is one or more of the markers selected from the group consisting of survivin, a mutant K-ras gene, and carcinoembryonic antigen.

38. The method of claim 36, wherein the sample is taken from at least one source selected from the group consisting of blood, urine, pancreatic juice, ascites, needle biopsy or tissue.

39. The method of claim 38, wherein the tissue is a frozen section.

40. The method of claim 36, wherein the quantification of the presence or progression of the pancreatic cancer is accomplished by FACS-scan analysis.

41. The method of claim 36, wherein the olignucleotide targets the pancreatic cancer marker survivin.

42. The method of claim 41, wherein the oligonucleotide is one or more selected from the group consisting of SEQ ID NOS: 1, 2 and 9.

43. The method of claim 36, wherein the oligonucleotide targets a mutant K-ras pancreatic cancer marker.

44. The method of claim 43, wherein the oligonucleotide is one or more selected from the group consisting of SEQ ID NOS: 7, 8, 11, 12 and 13.

45. A method of detecting cancerous cells in a sample comprising:
i) providing a sample of cells for analysis;
ii) treating the sample with an oligonucleotide that targets a cancer-specific marker gene sequence, wherein the oligonucleotide comprises at least one linked energy donor moiety and at least one linked energy acceptor moiety, wherein said oligonucleotide forms a stem-loop hairpin and wherein said donor and acceptor moieties are separated by at least a portion of a probing nucleobase sequence;
iii) detecting, identifying or quantitating the hybridization of the target oligonucleotide sequence to the pancreatic cancer marker gene sequence under suitable hybridization conditions, wherein the presence, absence or amount of target oligonucleotide sequence present in the sample is correlated with a change in detectable signal associated with at least one donor or acceptor moiety of the oligonucleotide; and
iv) detecting, identifying or quantitating the presence cancerous cells based upon the presence, absence or amount of the hybridization of the oligonucleotide to the cancer-specific target sequence that is determined.

46. The method of claim 45, wherein the quantification of the presence of a cancer cell is accomplished by FACS-scan analysis.

47. The method of claim 45, wherein the cancer cell originates from one or more of the cancers selected from the group consisting of: breast, pancreas, ovarian, prostate, colorectal, hepatocellular, multiple myeloma, lymphoma, bladder, medullary carcinoma of the thyroid, neuroendocrine tumors, carcinoid tumors, testicular, gestational trophoblast neoplasms, lung, melanoma and stomach.

48. A diagnostic kit for detecting or monitoring the progession of cancerous cells comprising materials suitable for carrying out the method of claim 36.

49. A diagnostic kit for detecting alterations in gene expression in viable cells in real-time comprising materials suitable for carrying out the method of claim 22.


SEQUENCE LISTING (13 sequences)

SEQ ID NO: 1 - DNA, 26 bp, Homo sapiens
  misc_feature (1)..(1): n is a, c, g, t or u
nctgagaaagggctgccagtctcagn

SEQ ID NO: 2 - DNA, 24 bp, Homo sapiens
ctgagaaagggctgccagtctcag

SEQ ID NO: 3 - DNA, 23 bp, Homo sapiens
tggagttgtcggtgtagactcca

SEQ ID NO: 4 - DNA, 23 bp, Homo sapiens
cacttgatcactctggacaagtg

SEQ ID NO: 5 - DNA, 22 bp, Homo sapiens
tagaggtggcggagcatctcta

SEQ ID NO: 6 - DNA, 22 bp, Homo sapiens
caatccgcagcctctgcgattg

SEQ ID NO: 7 - DNA, 22 bp, Homo sapiens
cctacgccatcagctccgtagg

SEQ ID NO: 8 - DNA, 22 bp, Homo sapiens
cctacgccaacagctccgtagg

SEQ ID NO: 9 - DNA, 23 bp, Homo sapiens
tggtccttgagaaagggcgacca

SEQ ID NO: 10 - DNA, 23 bp, Homo sapiens
gagtccttccacgataccgactc

SEQ ID NO: 11 - DNA, 22 bp, Homo sapiens
cctacgccacgagctccgtagg

SEQ ID NO: 12 - DNA, 22 bp, Homo sapiens
cctacgccacaagctccgtagg

SEQ ID NO: 13 - DNA, 22 bp, Homo sapiens
cctacgccagcagctccgtagg